(19) United States
(12) Patent Application Publication
(10) Pub. No.: US 2017/0320921 A1
(43) Pub. Date: Nov. 9, 2017

(54) PROTEIN M RELATED IMMUNOGLOBULIN-BINDING POLYPEPTIDES

(71) Applicants: Rajesh GROVER, Del Mar, CA (US); Richard LERNER, La Jolla, CA (US); Ian WILSON, La Jolla, CA (US); Xueyong ZHU, San Diego, CA (US); THE SCRIPPS RESEARCH INSTITUTE, La Jolla, CA (US)

(72) Inventors: Rajesh Grover, Del Mar, CA (US); Richard Lerner, La Jolla, CA (US); Ian Wilson, La Jolla, CA (US); Xueyong Zhu, San Diego, CA (US)

(21) Appl. No.: 15/115,773

(22) PCT Filed: Feb. 2, 2015
(86) PCT No.: PCT/US15/14055

§ 371 Date: Aug. 1, 2016

Related U.S. Application Data
(60) Provisional application No. 61/934,116, filed on Jan. 31, 2014.

(51) Int. Cl. C07K 14/30; C07K 1/22; C07K 16/00

(52) U.S. Cl. CPC C07K 14/30; C07K 16/00; C07K 1/22; C07K 2319/00

(57) ABSTRACT

The invention relates to a group of isolated polypeptides and derivatives that can bind genetically to immunoglobulins or antibodies. The invention also relates to industrial and other applications of these molecules, e.g., antibody purifications.

### CROSS-REFERENCE TO RELATED APPLICATIONS

The subject patent application claims the benefit of priority to U.S. Provisional Patent Application No. 61/934,116 (filed Jan. 31, 2014). The full disclosure of the priority application is incorporated herein by reference in its entirety and for all purposes.

### BACKGROUND OF THE INVENTION

Antibody purification processes have in general been relying on the use of gel electrophoresis, dialysis and chromatography, i.e., ion-exchange, gel filtration, hydroxylapatite chromatography, and affinity chromatography in particular. For example, Protein A is often used in affinity chromatography for capturing antibodies, which is followed by ion-exchange and/or hydrophobic interaction and/or mixed mode chromatography steps. Protein A is a 40-60 kDa surface protein originally found in the cell wall of the bacteria Staphylococcus aureus. It has found use in biochemical research because of its ability to bind immunoglobulins, most notably IgG's. It binds to the Fc region of immunoglobulins through interaction with the heavy chain.

There is a need in the art for alternative and more efficient means for purifying antibodies and antigen-binding molecules. The present invention is directed to this and other unmet needs in the art.

### SUMMARY OF THE INVENTION

In one aspect, the invention provides isolated or recombinant polypeptides which have an amino acid sequence that (a) is substantially identical to Protein MG281 having an amino acid sequence shown in SEQ ID NO:1 or to a Protein MG281 fragment, and (b) further contains deletion of the C-terminal domain or substitutions at one or more conserved residues for forming hydrogen bonds or salt bridge with antibodies. Typically, these polypeptides are capable of generically binding to immunoglobulins. In some embodiments, the conserved residues that can be modified include residues Ser106, Thr110, Tyr144, Tyr158, Ser160, Asn177, Arg384, Ala391, Asn440, and Tyr444. In some of these embodiments, the conserved residues are substituted with non-polar amino acid residues.

In various embodiments, the polypeptides of the invention have an amino acid sequence that (a) is at least 80%, 90%, 95% or 99% identical to Protein MG281 or fragment thereof, and (b) further contains the noted deletion or substitutions. Some of the polypeptides consist of an amino acid sequence that is identical to the sequence of Protein MG281 or fragment thereof, except for said deletion or substitutions. Some of the polypeptides of the invention have an amino acid sequence that harbors the noted amino acid substitutions, and that is otherwise identical or substantially identical a Protein MG218 fragment consisting of residues 37-556, residues 37-482, residues 74-482, residues 37-468, residues 74-468, residues 37-442, or residues 74-442 of SEQ ID NO:1. In some embodiments, the polypeptide consists essentially of an amino acid sequence that harbors an amino acid substitution at residue Y158 or 8384 and is otherwise identical to residues 74-482 of SEQ ID NO:1. In these embodiments, the amino acid substitution can be, e.g., Y158F or R384A.

In a related aspect, the invention provides isolated or recombinant soluble polypeptides that are derived from a protein shown in any one of SEQ ID NOs:18-33. These derivatives lack the N-terminal membrane-spanning region and are capable of generically binding to immunoglobulins. In some embodiments, the derivative polypeptides consist essentially of an amino acid sequence that is identical or substantially identical to an amino acid sequence shown in any one of SEQ ID NOs:18-33 minus the membrane-spanning region. Some of these polypeptides consist essentially of SEQ ID NO:22, 32 or 33. The derivative polypeptides can further have a deletion of the C-terminal domain. The derivative polypeptides can also harbor at least one amino acid substitution at the conserved residues responsible for hydrogen bond or salt bridge formation.

In another aspect, the invention provides isolated or recombinant soluble polypeptides that have an amino acid a sequence that (a) is substantially identical to a Protein M homolog or ortholog sequence selected from SEQ ID NOs:18-33 or fragment thereof, and (b) contains substitutions at one or more conserved residues for forming hydrogen bonds or salt bridge with antibodies. In various embodiments, these polypeptides are capable of generically binding to immunoglobulins. In some embodiments, the Protein M homolog or ortholog sequence lacks the N-terminal membrane-spanning region. In some embodiments, the Protein M homolog or ortholog sequence has a deletion of the C-terminal domain. In some embodiments, the Protein M homolog or ortholog sequence is SEQ ID NO:22 or SEQ ID NO:33, and the conserved residues are Tyr149, Ser111, Thr115, Asn456, Tyr459, Ala406, Tyr115, and Ser163. In some embodiments, the Protein M homolog or ortholog sequence is SEQ ID NO:32, and the conserved residues are Ala343, Tyr115, and Ser118.

In another aspect, the invention relates to methods of purifying or isolating immunoglobulin molecules via their binding to a Protein M variant that is derived from a protein shown in SEQ ID NOs:1 and 18-33, or a fragment thereof, and that is capable of generically binding to immunoglobulins. Such methods involve contacting the immunoglobulin-binding protein or fragment attached to a solid support with a biological sample containing the immunoglobulins for a time sufficient to allow the immunoglobulins to bind the immunoglobulin-binding protein or fragment thereof, and then eluting the immunoglobulin molecules from the solid support-attached protein or fragment thereof. In particular embodiments, the solid support can be agarose, polyacrylamide, dextran, cellulose, polysaccharide, nitrocellulose, silica, alumina, aluminum oxide, titania, titanium oxide, zirconia, styrene, polyvinyldifluoride nylon, copolymer of styrene and divinylbenzene, polymethacrylate ester, derivatized azlactone polymer or copolymer, glass, or cellulose; or a derivative or combination thereof. In a related aspect, the invention provides kits for using the immunoglobulin-binding proteins or fragments described herein in the purification of antibodies from various biological samples.

In other aspects, the invention provides polynucleotide sequences that encode the immunoglobulin-binding proteins or fragments thereof, as well as vectors harboring such polynucleotide sequences.

### DESCRIPTION OF THE DRAWINGS

FIG. 1 shows the amino acid sequence of Mycoplasma genitalium MG281 protein as provided in GenBank Accession No. P47523.1 (SEQ ID NO:1).

FIG. 2 shows the amino acid sequence of a soluble form of MG281 (i.e., amino acid residues 37-556 of SEQ ID NO:1) with an N-terminal 6-His tag, followed by a thrombin cleavage site (both in bold) (SEQ ID NO:2).

FIG. 3 shows the amino acid sequences of the light chain and heavy chain from the crystal structure of the Fab fragment of the immunoglobulin purified from multiple myeloma patient plasma sample 13PL. Also shown are the CDR sequences present in each chain.

FIG. 4 shows amino acid sequences for a trypsin digested Protein M (“Protein M TD” or “MG281-T”) (which contains amino acid residues 74 to 468 of SEQ ID NO:1)(SEQ ID NO:11); and amino acid sequences for two fragments of MG281: F1 (which contains amino acid residues 134 to 269 of SEQ ID NO:1) (SEQ ID NO:12), and F2 (which contains amino acid residues 279 to 452 of SEQ ID NO:1) (SEQ ID NO:13). All three sequences shown (SEQ ID NOs:15-17) additionally have an N-terminal 6-His tag and thrombin cleavage site (both in bold), thereby showing the recombinant MG281 constructs.

FIGS. 5A-5D show that immunoglobulins selectively bind to proteins in human mycoplasma. (A) (Left panel) Western blot analysis of the reactivity of plasma from multiple myeloma patient 13PL with cell extracts from Mycoplasma alligatoris, Mycoplasma crocodyli, Mycoplasma fermentans, M genitalium, Acholeplasma laidlawii, Mycoplasma mycoides, Mycoplasma penetrans, Mycoplasma pneumoniae and Mycoplasma pulmonis. All mycoplasma cells were grown in appropriate media. Cells were lysed according to manufacturer's protocol using lysis buffer from Sigma Aldrich. Nucleic acids were degraded by treatment with DNAase and RNAase. A protease inhibitor cocktail (Roche) was added to prevent proteolytic degradation. The extracts from the same number of cells were separated on SDS-PAGE gels and transferred to nitrocellulose membranes for Western blot analysis. (Right panel) Ponceau red-stained protein bands of the cell extracts. (B) Crystals of 13PL Fab′ from a multiple myeloma patient's monoclonal immunoglobulin. (C) Western blot analysis of the reactivity of 13PL Fab′ from the plasma of a multiple myeloma patient with the same cell extracts in A. The 13PL Fab′ was purified by crystallization. The extracts were separated on SDS-PAGE gels as described in (A). (D) Crystal structure of 13PL Fab′ shown in ribbon diagram with the light and heavy chains.

FIG. 6 shows comparison of Protein M and Protein M TD mutants' reactivity with multiple myeloma (IgG) antibody. Western blot analysis of the Multiple Myeloma antibody reactivity (13PL) with affinity purified recombinant Protein M TD mutants PM2, PM3, PM4, PM5, PM6 and PM7 at the same concentration (1 μg/well). The proteins were separated on SDS-PAGE gel and transferred to nitrocellulose membranes for Western blot.

FIG. 7 shows confirmation and comparison of two Protein M TD mutant's reactivity with multiple myeloma (IgG) antibody. Western blot analysis of the Multiple Myeloma antibody reactivity (13PL) with affinity purified recombinant Protein M TD mutants PM1 and PM2 at the same concentration (1 μg/well). The proteins were separated on SDS-PAGE gel and transferred to nitrocellulose membranes for Western blot.

FIG. 8 shows confirmation and comparison of binding to multiple myeloma antibodies by Protein M homolog from Mycoplasma pneumonia and Protein M variants from Mycoplasma genitalium (MG281). Western blot analysis of the Multiple Myeloma antibody reactivity (13PL) with affinity purified recombinant Mpn400 (residues 75-484, 1 μg), Mpn400 (residues 41-582, 1 μg), Protein M TD (residues 74-442, 1 μg) (truncated) and Protein M (residues 37-556, 2 μg) (full length). The proteins were separated on SDS-PAGE gel and transferred to nitrocellulose membranes for Western blot.

FIG. 9 shows confirmation of immunoglobulin binding protein from Mycoplasma penetrans reactivity with multiple myeloma (IgG) antibody. The proteins were separated on SDS-PAGE gel and transferred to nitrocellulose membranes for Western blot. Shown in the middle lanes of the figure are the results of Western blot analysis of the 13PL antibody reactivity with affinity purified recombinant protein MYPE1380 (residues 41-503) in the amounts of 1 μg/well and 1.5 μg/well, respectively.

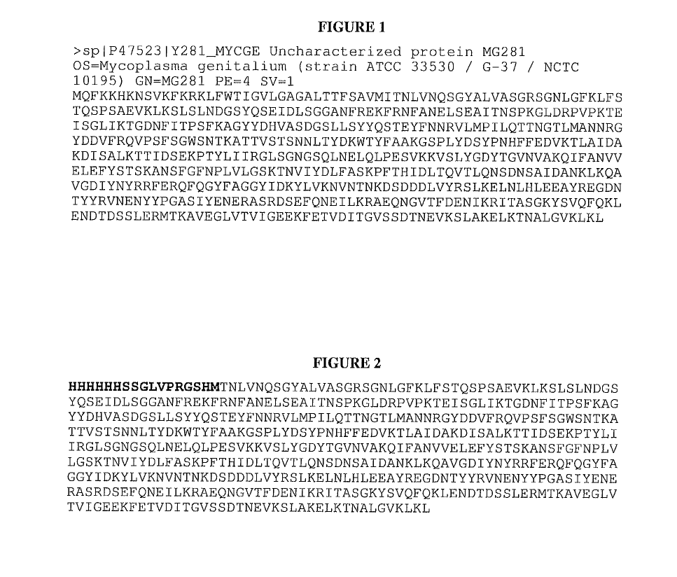

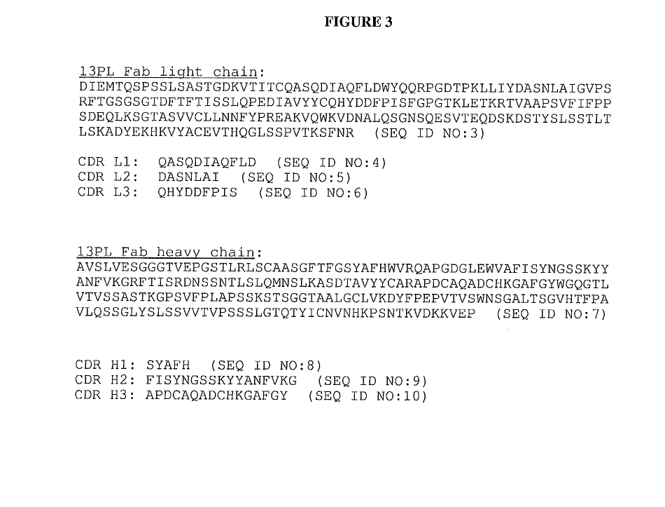

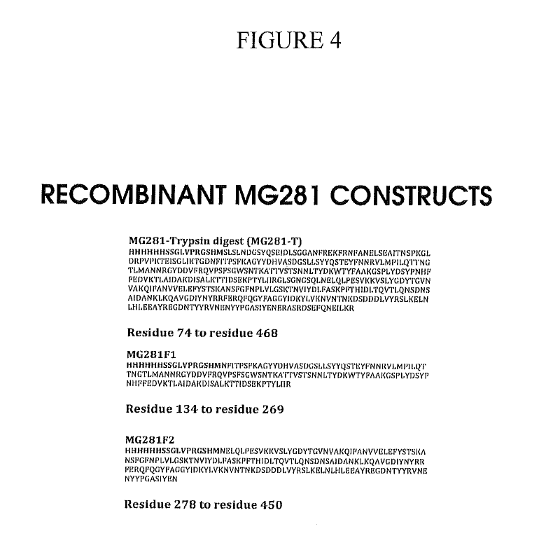

### DETAILED DESCRIPTION OF THE INVENTION

### I. Overview

Mycoplasma genitalium protein MG281 (Protein M) has the properties of a class of non-specific immunoglobulin binding proteins, sometimes referred to as B-cell super antigens. Protein M binds to immunoglobulins and blocks reactivity of the antibody with its cognate antigen. It is about 50 kDa in size, and composed of 556 amino acids. Protein M has a large domain of 360 amino acid residues that binds primarily to the variable light chain of the immunoglobulin, as well as a binding site called LRR-like motif. It also has a C-terminal domain with 115 amino acid residues that protrudes over the antibody binding site. Proteomics analysis showed that Protein M additionally contained a 16-36 amino acid transmembrane domain.

The invention is predicated on the identification by the present inventors of a number of Protein M homologs or orthologs from mycoplasmas and other species that share functional and structural properties with protein MG281. The inventors also developed several specific variants and orthologs of Protein M that have similar or improved Ig-binding properties. For example, the inventors demonstrated that Protein M variants lacking the C-terminal domain retains the ability to bind to immunoglobulins. The inventors also demonstrated that variants with amino acid substitutions at one or more of the conserved residues for forming hydrogen bonds with antibodies can have improved immunoglobulin-binding properties. For example, it was found that alteration at one or more of these consensus residues (e.g., residues Tyr158 and Arg384) can lead to Protein M variants with decreased binding affinities, which allow better antibody elution when the variants are used in antibody purification.

Importantly, the immunoglobulin-binding polypeptides of the invention (e.g., Protein M) binds to antibodies with either κ or λ light chains using conserved hydrogen bonds and salt bridges from backbone atoms and conserved side chains, and some conserved van der Waals interactions, as well as other non-conserved interactions. These conserved interactions provide a structural basis for the broad reactivity with Fvs, Fabs or Igs. This is in contrast to Protein G and Protein A, which have their primary binding site in the antibody Fc domain. Thus, an apparent advantage of the immunoglobulin-binding proteins of the invention is that they are suitable for purifying antibody fragments that do not contain the Fc domain, e.g., scFv fragments and Fab fragments.

The invention accordingly provides a series of Protein M variants that (1) are derived from protein MG281 and other Protein M orthologs or homologs, and (2) are capable of generically binding to antibodies or immunoglobulins. Relative to the full length wildtype sequences, sequences of the Protein M variant polypeptides of the invention can be substantially identical to the wildtype sequences or fragments thereof. The sequences can also contain sequence deletions, e.g., deletion of N-terminal membrane spanning region or the C-terminal domain. The sequence can also contain substitutions at various locations, e.g., substitutions of the hydrogen bond-forming conservative residues with non-polar amino acid residues. The invention also provides related methods and kits of using the Protein M variants for purifying antibodies or immunoglobulins.

Unless otherwise specified, numbering of amino acid residues in Protein M or variants or orthologs (e.g., the consensus residues for hydrogen bonding) is based on the prototype Protein M molecule from M. genitalium (aka Protein MG281). As detailed herein, consensus residues in Protein MG281 responsible for the binding activities include, e.g., 5106, TI 10, Y144, Y158, S160, R384, A391, N440, and Y444. As exemplified herein for Protein M homologs Mpn400 and MYPE1380, corresponding residues in other Protein M orthologs or variants can be easily ascertained via, e.g., sequence alignment. In addition, hydrogen bonding and salt bridge interactions between a Protein M homolog or ortholog and antibodies can be assessed with methods known in the art, e.g., McDonald et al., J. Mol. Biol. 238, 777-793, 1994; and Sheriff et al., J. Mol. Biol. 197, 273-296, 1987.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3PrdP ed., 2002); and A Dictionary of Biology (Oxford Paperback Reference), Martin and Hine (Eds.), Oxford University Press (4PthP ed., 2000). In addition, the following definitions are provided to assist the reader in the practice of the invention.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “amino acid” of a peptide refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The Protein M related polypeptides of the invention encompass derivatives or analogs which have been modified with non-naturally coding amino acids.

The terms “antibody” (Ab) and “immunoglobulin” (Ig) are used interchangeably herein and refer to a large generally Y-shaped protein produced by B-cells that is used by the immune system to identify and neutralize foreign objects such as bacteria and viruses. Antibodies are typically made of basic structural units, each with two large heavy chains and two small light chains. There are several different types of antibody heavy chains, and several different kinds of antibodies, which are grouped into different isotypes based on which heavy chain they possess. There are five types of mammalian Ig heavy chain, which defines the class of antibody, and are denoted by the Greek letters: α, δ, ε, γ, and μ, found in IgA, IgD, IgE, IgG, and IgM antibodies, respectively. Each heavy chain has two regions, the constant region and the variable region. The constant region is identical in all antibodies of the same isotype, but differs in antibodies of different isotypes. The variable region of the heavy chain differs in antibodies produced by different B cells, but is the same for all antibodies produced by a single B cell or B cell clone. In mammals there are two types of immunoglobulin light chain, which are called lambda (λ) and kappa (κ). A light chain has two successive domains: one constant domain and one variable domain. Each antibody contains two light chains that are always identical.

Antibodies can occur in two physical forms, a soluble form that is secreted from the cell, and a membrane-bound form that is attached to the surface of a B cell and is referred to as the B cell receptor (BCR). The BCR is only found on the surface of B cells and facilitates the activation of these cells and their subsequent differentiation into either antibody factories called plasma cells, or memory B cells that will survive in the body and remember that same antigen so the B cells can respond faster upon future exposure to the antigen. Secreted antibodies are produced by plasma cells.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multi-specific antibodies (e.g., bispecific antibodies). As used herein, the term “antibody fragment” of an antibody (the “parental antibody”) encompasses a fragment or a derivative of an antibody, typically including at least a portion of the antigen binding or variable regions (e.g. one or more CDRs) of the parental antibody, that retains at least some of the binding specificity of the parental antibody.

Examples of antibody fragments include, but are not limited to, Fab, Fab′, F(ab′)₂, and Fv fragments, diabodies, linear antibodies, single-chain antibody molecules, e.g., scFv; and multi-specific antibodies formed from antibody fragments. Typically, a binding fragment or derivative retains at least 10% of parental antibody's binding activity when that activity is expressed on a molar basis. Preferably, a binding fragment or derivative retains at least 20%, 50%, 70%, 80%, 90%, 95% or 100% or more of the antigen binding affinity as the parental antibody. It is also intended that a binding fragment can include conservative amino acid substitutions (referred to as “conservative variants” of the antibody) that do not substantially alter its biologic activity.

A “Fab fragment” is composed of one light chain and the C_{H}1 and variable regions of one heavy chain.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” contains the variable regions from both the heavy and light chains, but lacks the constant regions.

The term “single-chain Fv” or “scFv” antibody refers to antibody fragments containing the V_{H} and V_{L} domains of an antibody, wherein these domains are present in a single polypeptide chain. Generally, the Fv polypeptide further contains a polypeptide linker between the V_{H} and V_{L} domains, which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The term “monoclonal antibody”, as used herein, refers to an antibody obtained from a population of substantially homogeneous antibodies, i.e., the individual antibodies constituting the population are identical except for possible naturally occurring mutations that may be present in minor amounts.

As used herein, the term “diabodies” refers to small antibody fragments with two antigen-binding sites, which fragments contain a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., Holliger et al. (1993) Proc. Natl. Acad. Sci. USA 90: 6444-6448. For a review of engineered antibody variants generally, see Holliger and Hudson (2005) Nat. Biotechnol. 23:1126-1136.

As used herein, the term “humanized antibody” refers to forms of antibodies that contain sequences from both human and non-human (e.g., bovine, goat, murine, and rat) antibodies. In general, the humanized antibody will contain substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

As used herein, the term “hypervariable region” refers to the amino acid residues of an antibody which are responsible for antigen-binding. The hypervariable region comprises amino acid residues from a “complementarity determining region” or “CDR” and/or those residues from a “hypervariable loop” in the light chain variable domain and in the heavy chain variable domain. As used herein, the term “framework” or “FR” residues refers to those variable domain residues other than the hypervariable region residues defined herein as CDR sequences.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention apparatus and methods of use thereof corresponding to the scope of each of these phrases. Thus, an apparatus or method comprising recited elements or steps contemplates particular embodiments in which the apparatus or method consists essentially of or consists of those elements or steps.

The term “conservatively modified variant” applies to both amino acid and nucleic acid sequences. For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “fragment” refers to any peptide or polypeptide having an amino acid residue sequence shorter than that of a full-length polypeptide whose amino acid residue sequence is described herein. Relative to a full length Protein M homolog or ortholog sequence, some of the Protein M variants comprise a fragment sequence that has truncation at the N-terminus to remove the membrane domain. These fragments can additionally contain C-terminus truncations (e.g., truncations of up to 10, 20, 30, 50, 100 or more C-terminal residues).

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

The terms “nucleic acid,” “nucleic acid molecule,” or “nucleic acid fragment” refers to any one or more nucleic acid segments, e.g., DNA or RNA fragments, present in a polynucleotide or construct. While the term “nucleic acid,” as used herein, is meant to include any nucleic acid, the term “nucleic acid fragment” is used herein to refer to a fragment of nucleic acid molecule encoding a polypeptide, or fragment, variant, or derivative thereof. As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, and the like, are not part of a coding region. Two or more nucleic acids or nucleic acid fragments of the present invention can be present in a single polynucleotide construct, e.g., on a single plasmid, or in separate polynucleotide constructs, e.g., on separate plasmids. Furthermore, any nucleic acid or nucleic acid fragment may encode a single polypeptide, e.g., a single antigen, an antibody or antibody fragment, a cytokine, or regulatory polypeptide, or may encode more than one polypeptide, e.g., a nucleic acid may encode two or more polypeptides. In addition, a nucleic acid may encode a regulatory element such as a promoter or a transcription terminator, or may encode heterologous coding regions, e.g. specialized elements or motifs, such as a secretory signal peptide or a functional domain.

The term “isolated” means the protein is removed from its natural surroundings. However, some of the components found with it may continue to be with an “isolated” protein. Thus, an “isolated polypeptide” is not as it appears in nature but may be substantially less than 100% pure protein.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, Wis.); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A mitogen is a chemical substance that encourages a cell to commence cell division, triggering mitosis. A mitogen is usually some form of a protein. Mitogenesis is the induction (triggering) of mitosis, typically via a mitogen. Mitogens trigger signal transduction pathways in which mitogen-activated protein kinase (MAPK) is involved, leading to mitosis.

As used herein, the term “orthologs” or “homologs” refers to polypeptides that share substantial sequence identity and have the same or similar function from different species or organisms. For example, Protein M homologs from different human Mycoplasma species are orthologs due to the similarities in their sequences and functions

As used herein, the term “variant” refers to a molecule (e.g., a polypeptide or polynucleotide) that contains a sequence that is substantially identical to the sequence of a reference molecule. For example, the reference molecule can be an N-terminally truncated MG281 polypeptide (as shown in SEQ ID NO:2) or a polynucleotide encoding the polypeptide. In some embodiments, the variant can share at least 50%, at least 70%, at least 80%, at least 90, at least 95% or more sequence identity with the reference molecule. In some other embodiments, the variant differs from the reference molecule by having one or more amino acid substitutions at conserved residues. In some other embodiments, a variant of a reference molecule has altered amino acid sequences (e.g., with one or more conservative amino acid substitutions) but substantially retains the biological activity of the reference molecule. Conservative amino acid substitutions are well known to one skilled in the art.

### III. Protein M Variants and Related Methods

Provided in the invention are isolated or recombinant proteins or fragments thereof (e.g., the soluble portion) that are derived from MycoplasmaMG281 or its homologs described herein (e.g., SEQ ID NO:2 and SEQ ID NOs:18-33). These isolated or recombinant proteins or fragments are capable of generically binding to immunoglobulins. As described herein, the term “generically binding to immunoglobulins” refers to a high affinity but non-specific binding to immunoglobulins in general as opposed to a specific binding to a specific antibody that is immune-reactive with a cognate antigen. In particular embodiments the MG281 derived proteins consist of an amino acid sequence shown in SEQ ID NO: 2; residues 18-537 of SEQ ID NO:2 (SEQ ID NO:14); or an amino acid sequence shown in SEQ ID NO:11, 12, or 13. Some other isolated or recombinant proteins or fragments of the invention are derived from a protein shown in any one of SEQ ID NOs:18-33. In some of these embodiments, the polypeptide or fragment thereof comprises or consists of an amino acid sequence that is identical or substantially identical to any one of SEQ ID NOs:18-33. In some other embodiments, the polypeptide or fragment thereof comprises or consists of the immunoglobulin-binding domain or portion of the protein shown in any one of SEQ ID NOs:18-33.

The invention also provides variant Protein M molecules that retain the ability to generically bind to immunoglobulins. These include variants of Protein MG281 or other Protein M homologs orthologs with various modifications, e.g., conservative substitutions at one or more residues. In some embodiments, the modifications are at one or more of the consensus residues that are responsible for hydrogen bond or salt bridge formations. In some embodiments, these consensus residues are modified via conservative substitutions. In some other embodiments, the consensus residues are substituted with nonpolar amino acid residues, e.g., S106A, Y144F, Y158F, S160A, R384A, R384K, Y444F. For example, the variants can have substitutions at 1, 2, 3, 4, 5, 6 or more of these consensus residues. In some embodiments, the variant Protein M molecules of the invention are deletion mutants. As exemplified herein for Protein MG281, such mutants include Protein M variants which have part or all of the C-terminal domain deleted. In some embodiments, the Protein M variants contain modifications that result in reduced binding affinity for immunoglobulins. For example, relative to binding affinity of MG218, the variants can have a binding dissociation constant that is at least 15%, 25%, 50%, 75%, 100%, 200%, 300%, 400%, or 500% higher.

The exemplified MG281 protein variants, orthologs or fragments capable of generically binding to immunoglobulins, and other Protein M variants or derivatives (e.g., polypeptide fragments) capable of generically or non-specifically binding to immunoglobulins can all be obtained in accordance with routine immunological and biochemical methods well known in the art or the specific assays exemplified herein. Thus, polypeptide fragments derived from MG281 or any one of SEQ ID NOs:18-33 can be readily generated via routinely practiced methods, e.g., recombinant expression. The polypeptide fragments can then be examined for ability to generically bind to immunoglobulins. Immunoglobulin-binding activity of a polypeptide fragment derived from Protein M (SEQ ID NO:1) or proteins shown in SEQ ID NOs:18-33 can be examined using methods well known in biochemistry and immunochemistry, e.g., the specific assays exemplified herein. In some embodiments, the Protein M variants or immunoglobulin-binding fragments derived from any one of the proteins shown in SEQ ID NOs:1 and 18-33 can contain at least 25, 50, 60, 70, 80, 90, 100, 150, 200, 250, 300, 350, 400, 450 or more contiguous amino acid residues in length. The sequences of these polypeptide fragments can be identical or substantially identical (e.g., at least 75%, 80%, 85%, 90%, 95% or 99% identical) to the corresponding contiguous amino acid residues of any one of SEQ ID NOs:1 and 18-33.

In some embodiments, the invention provides variant Protein M variant polypeptides that are derived from Mycoplasma genitalium MG281 protein (SEQ ID NO:1) or MG281 homologs or orthologs described herein. Typically, these Protein M variants have an amino acid sequence that is substantially identical (e.g., at least 60%, 70%, 75%, 80%, 90%, 95% or 99% identical) to the sequence of the MG281 protein or a fragment of the MG281 protein, and are capable of generically binding to immunoglobulins. Preferably, the variant polypeptides are soluble proteins derived from MG281, e.g., lacking the N-terminal membrane-spanning region. The MG281 derived Protein M variants can additionally contain a sequence alteration relative to the sequence of MG281 protein. Some of these variant polypeptides contain a deletion, e.g., a deletion of part or all of the C-terminal domain. Some of these variant polypeptides contain a deletion of N-terminal residues beyond the membrane-spanning region, e.g., N-terminal truncation up to residue 74. Some of these variant polypeptides contain a partial deletion of the C-terminal domain, e.g., C-terminal truncation up to residue 468 or 482. In various embodiments, the Protein M variants can contain a sequence that is identical or substantially identical to a MG281 fragment, e.g., residues 37-556, residues 37-482, residues 74-482, residues 37-468, residues 74-468, residues 37-442, or residues 74-442 of the full length MG281 sequence (SEQ ID NO:1).

Other than deletions, some of the MG281 derived Protein M variants can alternatively or additionally contain amino acid substitutions, including conservative substitutions at one or more residues. In some of these embodiments, the substitutions are at conserved residues in MG281 that are responsible for forming hydrogen bonds or salt bridge with antibodies or immunoglobulins. For example, the amino acid substitutions can be at one or more of these conserved residues, Ser106, Thr110, Tyr144, Tyr158, Ser160, Arg384, Ala391, Asn440, and Tyr444. In some embodiments, the conserved residues are substituted with non-polar amino acid residues, e.g., Ala or Phe as exemplified herein. In some of these embodiments, the substitutions lead to decreased hydrogen bond formation between the protein and antibodies. As exemplified herein, two examples of MG281 derived Protein M variants are PM2 and PM5, which consists of residues 74-482 of MG281 except for amino acid substitution Y158F or R384A, respectively.

The inventors also identified a number of Protein M homologs or orthologs from mycoplasmas and other species. As demonstrated in the Examples below, these orthologs or homologs can be similarly employed in the various industrial applications described herein. Typically, the Protein M orthologs or homologs suitable for the invention have an amino acid sequence that is substantially identical to the sequence exemplified herein (e.g., SEQ ID NOs:18-33) and are capable of generically binding to immunoglobulins. In some embodiments, the N-terminal membrane spanning region is removed from the wildtype protein sequences (e.g., SEQ ID NOs:22-33). For the other exemplified full length protein M homolog or ortholog sequences (e.g., SEQ ID NOs:18-21), the membrane spanning region can be readily determined via sequence alignment and other routinely used bioinformatics tools. In various embodiments, at least the first 15, 16, 17, 18, 19, 20, 25, 30, 35, 40, 50, 60, 70 or more N-terminal residues can be removed from these sequences. Some of Protein M orthologs or homologs suitable for the invention consist essentially of an amino acid sequence shown in SEQ ID NO:22, 32 or 33. Relative to the wildtype sequences (e.g., SEQ ID NOs:18-33), the Protein M ortholog or homolog polypeptides can also contain additional sequence deletions, e.g., part or all of the C-terminal domain. They can also harbor one or more amino acid substitutions at the conserved residues responsible for hydrogen bond or salt bridge formation.

Some specific Protein M homologs, or Protein M homologs minus the transmembrane domain, that can be utilized in the practice of the invention include, e.g., CM1_01690 (Mycoplasma genitalium M6320; YP_006600814.1; SEQ ID NO:18), CM5_01645 (Mycoplasma genitalium M2288; YP_006601319; SEQ ID NO:19); CM9_01665 (Mycoplasma genitalium M2321; YP_006599823; SEQ ID NO:20); CM3_01775 (Mycoplasma genitalium M6282; YP_006600310; SEQ ID NO:21); MPN400 (Mycoplasma pneumoniae M129; NP 110088; SEQ ID NO:22), G4EN64 (Mycoplasma iowae; WP 004025288; SEQ ID NO:23), R8B750 (Mycoplasma gallisepticum; WP_011884082; SEQ ID NO:24), D3FGB7 (Mycoplasma gallisepticum str. R(high); YP_005879786; SEQ ID NO:25), Q7NBM4 (Mycoplasma gallisepticum str. R(low); NP_853026; SEQ ID NO:26), J3YUE1 (Mycoplasma gallisepticum NC06_2006.080-5-2P; YP_006584926; SEQ ID NO:27), J3YF71 (Mycoplasma gallisepticum NY01_2001.047-5-1P; YP_006583426; SEQ ID NO:28; J3T7A1 (Mycoplasma gallisepticum NC96_1596-4-2P; YP_006582653; SEQ ID NO:29), J3VAC2 (Mycoplasma gallisepticum VA94_7994-1-7P; YP_006581144; SEQ ID NO:30), D3FIQ8 (Mycoplasma gallisepticum str. F; YP_005880864; SEQ ID NO:31), and MYPE1380 (Q8EWR5) (Mycoplasma penetrans HF-2; NP_757525; SEQ ID NO:32). In addition, a shorter version of MPN400 contains residues 75-484 (SEQ ID NO:33). Amino acid sequences of these proteins are noted below.

| SEQ ID NO: 18 | |
|---|---|
| l | mqfkkhknsv kfkrklfwti gvlgagaltt fsavmitnlv nqsgyalvas grsgnlgfkl |
| 6l | fstqspsaev klkslslndg syqseidlsg ganfrekfrn fanelseait nspkgldrpv |
| l2l | pkteisglik tgdnfitpsf kagyydhvas dgsllsyyqs teyfnnrvlm pilqttngtl |
| l8l | mannrgyddv frqvpsfsgw sntkattast snnitydkwt yfaakgsply dsypnhffed |
| 24l | vktlaidakd isalkttids ekptyliirg lsgngsqlne lqlpesvkkv slygdytgvn |
| 30l | vakqifanvv elefystska nsfgfnplvl gsktnviydl faskpfthid ltqvtlqnsd |
| 36l | nsaidanklk qavgdiynyr rferqfqgyf aggyidkylv knvntnkdsd ddlvyrslke |
| 42l | lnlhleeayr egdntyyrvn enyypgasiy enerasrdse fqneilkrae qngvtfdeni |
| 48l | kritasgkys vqfqklendt dsslermtka veglvtvige ekfetvditg vssdtnevks |
| 54l | lakelktnal gvklkl |
| SEQ ID NO: 19 | |
| l | mqfkkhknsv kfkrklfwti gvlgagaltt fsavmitnlv nqsgyalvvp grsgnlgfkl |
| 6l | fstqspsaev klkslslndg syqseidlsg ganfrekfrn fanelseait nspkgldrpv |
| l2l | pkteisglik tgdnfitpsf kagyydhvas dgsllsyyqs teyfnnrvlm pilqttngtl |
| l8l | mannrgyddv frqvpsfsgw sntkattvst snnitydkwt yfaakgsply dsypnhffed |
| 24l | vktlaidakd isalkttids ekptyliirg lsgngsqlne lqlpesvkkv slygdytgvn |
| 30l | vakqifanvv elefystska nsfgfnplvl gsktnviydl faskpfthid ltqvtlqnsd |
| 36l | nsaidanklk qavgdiynyr rferqfqgyf aggyidkylv knvntnkdsd ddlvyrslke |
| 42l | lnlhleeayr egdntyyrvn enyypgasiy enerasrdse fqneilkrae qngvtfdeni |
| 48l | kritasgkys vqfqklendt dsslermtka veglvtvige ekfetvditg vssdtnevks |
| 54l | lakelktnal gvklkl |
| SEQ ID NO: 20 | |
| l | mqfkkhknsv kfkrklfwti gvlgagaltt fsavmitnlv nqsgyalvas grsgnlgfkl |
| 6l | fstqspsaea klkslslndg syqseidlsg ganfrekfrn fanelseait nspkgldrpv |
| l2l | pkteisglik tgdnfitpsf kagyydhvas dgsllsyyqs teyfnnrvlm pilqttngtl |
| l8l | mannrgyddv frqvpsfsgw sntkattvst snnitydkwt yfaakgsply dsypnhffed |
| 24l | vktlaidakd isalkttids ekptyliirg lsgngsqlne lqlpesvkkv slygdytgvn |
| 30l | vakqifanvv elefystska nsfgfnplvl gsktnviydl faskpfthid ltqvtlqnsd |
| 36l | nsaidanklk qtvgdiynyr rferqfqgyf aggyidkylv knvntnkdsd ddlvyrslke |
| 42l | lnlhleeayr egdntyyrvn enyypgasiy enerasrdse fqneilkrae qngvtfdeni |
| 48l | kritasgkys vqfqklendt dsslermtka veglvtvige ekfetvditg vssdtnevks |
| 54l | lakelktnal gvklkl |
| SEQ ID NO: 21 | |
| l | mqfkkhknsv kfkrklfwti gvlgagaltt fsavmitnlv nqsgyalvas grsgnlgfkl |
| 6l | fstqsssaev klkslslndg syqseidlsg ganfrekfrn fanelseait nspkgldrpv |
| l2l | pkteisglik tgdnfitpsf kagyydhvas dgsllsyyqs teyfnnrvlm pilqttngtl |
| l8l | mannrgyddv frqvpsfsgw sntkattvst snnitydkwt yfaakgsply dsypnhffed |
| 24l | vktlaidakd isalkttids ekptyliirg lsgngsqlne lqlpesvkkv slygdytgvn |
| 30l | vakqifanvv elefystska nsfgfnplvl gsktnviydl faskpfthid ltqvtlqnsd |
| 36l | nsaidanklk qavgdiynyr rferqfqgyf aggyidkylv knvntnkdsd ddlvyrslke |
| 42l | lnlhleeayr egdntyyrvn enyypgasiy enerasrdse fqneilkrae qngvtfdeni |
| 48l | kritasgkys vqfqklendt dsslermtka veglvtvige ekfetvditg vssdtnevks |
| 54l | lakelktnal gvklkl |
| SEQ ID NO: 22 | |
| 4l | nevlrlqsge tliasgrsgn |
| 6l | lsfqlyskvn qnaksklnsi sltdggyrse idlgdgsnfr edfrnfannl seaitdapkd |
| l2l | llrpvpkvev sgliktsstf itpnfkagyy dqvaadgktl kyyqsteyfn nrvvmpilqt |
| l8l | tngtltannr ayddifvdqg vpkfpgwfhd vdkayyagsn gqseylfkew nyyvangspl |
| 24l | ynvypnhhfk qiktiafdap rikqgntdgi nlnlkqrnpd yviingltgd gstlkdlelp |
| 30l | esvkkvsiyg dyhsinvakq ifcnvlelef ystnqdnnfg fnplvlgdht niiydlfask |
| 36l | pfnyidltsl elkdnqdnid asklkraysd iyirrrferq mqgywaggyi drylvkntne |
| 42l | knvnkdndtv yaalkdinlh leetythggn tmyrvnenyy pgasayeaer atrdsefqke |
| 48l | ivqraeligv vfeygvknlr pglkytvkfe spqeqvalks tdkfqpvigs vtdmsksvtd |
| 54l | ligvlrdnae ilnitnvskd etvvaelkek ldrenvfqei rt |
| SEQ ID NO: 23 | |
| 3l | qntsvnvnnn eninyktngt vvtgdkltfs |
| 6l | avvqqnsnis tqafisdgtk pvgtynkein lgkdsitpky tsgyvetyle sgdtvsryss |
| l2l | seyhnnrtlm pildtkehyy tsertyseiq kgiyrgweis tpsinygekf synasavlkt |
| l8l | vfkqlkqetv savqfnlgls dtsiesinsf lktnsdiqfv tikgisqdtd lsklvlpesv |
| 24l | qkltllgqrn tindlklpse lqeieiylgs slksidplif pksaniisdv vmnntssvft |
| 30l | eiklsdstid nnspklqeai ddvytyrike rafqglvpgg yiaswdltgt kvtsfnnvni |
| 36l | ppindgtgrf yiahvevktd gnfgnsqnes igskpsndsq indwfdwggg wqkvqevvvs |
| 42l | ssenvsleta tqeimgfiak ypnvkkiniv nvkltdgsth eqlkdnvika itakygeesq |
| 48l | ykdiefvlpe tvpspva |
| SEQ ID NO: 24 | |
| 4l | slyqdkqisg qnqplapvnr |
| 6l | lidfqtlakf riedldfelq kkiysstves aelvnrsavl vddsvlenhd geltsgqsdp |
| l2l | qvpapvkila keqtghtsdf vsgysddnky yqspyyyndr vympildspt iylknertss |
| l8l | diglnnyqgw iavgharvns rvsvfnyrat dellakfnnl pdrliftmli dlyqanpaii |
| 24l | netlkeyspd fvilsnadsq tmkqlvfpss vkkltiksni ldrfdfslvn seiqelelyt |
| 30l | pniteynpla lnpkthlifd adystrflsi nlygaqltnq qalaaledvf vhryyeralq |
| 36l | gsfvggyiss lvlsdtgits lnnlviknin pnydsytmsv kyhsndsgqi ellkttawkt |
| 42l | ptptptpdqa aapgdgrvsv edkdlrllvs sevpvnaevl invvskylyn ntrvnildis |
| 48l | ksllksgslv dvanslkaki pylnvvi |
| SEQ ID NO: 25 | |
| 3l | iytsvkisns lyqdkqisgq nqplapvnrl |
| 6l | igfqtlakfr iedldfelqk kiysstvesa elvnrsavlv ddsvlenhdg eltsgqsdpq |
| l2l | vpapvkilak eqtghtsdfv sgysddnkyy qspyyyndry ympildspti ylknertssd |
| l8l | iglnnyqgwi avgharvnsr vsvfnyratd ellakfnnlp drliftmsid lyqanpamin |
| 24l | etlkeyspdf vilsnadskt mkqlvfpssv kkltiksnil drfnfslyns eiqelelytp |
| 30l | niteynplal npkthlifda dystrflsin lygaqltnqq alaaledvfv hryyeralqg |
| 36l | sfvggyissl vlsdtgitsl nnlvikninp nydsytmsvk yhsndsgqie llkttawktp |
| 42l | tptptpdqaa apgdgrvnve dkdlrllvss evpvnaevli nvvskylynn trvnildisk |
| 48l | sllksgslvd vanslkakip ylnvvi |
| SEQ ID NO: 26 | |
| 4l | lyqdkqisgq nqplapvnrl |
| 6l | igfqtlakfr iedldfelqk kiysstvesa elvnrsavlv ddsvlenhdg eltsgqsdpq |
| l2l | vpapvkilak eqtghtsdfv sgysddnkyy qspyyyndry ympildspti ylknertssd |
| l8l | iglnnyqgwi avgharvnsr vsvfnyratd ellakfnnlp drliftmsid lyqanpamin |
| 24l | etlkeyspdf vilsnadskt mkqlvfpssv kkltiksnil drfnfslvns eiqelelytp |
| 30l | niteynplal npkthlifda dystrflsin lygaqltnqq alaaledvfv hryyeralqg |
| 36l | sfvggyissl vlsdtgitsl nnlvikninp nydsytmsvk yhsndsgqie llkttawktp |
| 42l | tptptptptp tpdqaaapgd grvnvedkdl rllvssevpv naevlinvvs kylynntrvn |
| 48l | ildisksllk sgslvdvans lkakipylnv vi |
| SEQ ID NO: 27 | |
| 3l | iytsvkisns lyqdklisgq nqplapvnrl |
| 6l | igfqmlakfr iedldfelqk kiysstvesa elvnrsavlv ddsvlenhdg eltsvqshpq |
| l2l | vpapvkilak eqtghtsdfv sgysddnkyy qspyyyndry ympildspti ylknertssd |
| l8l | iglnnyqgwi avgharvnsr vsvfnyratd ellakfnnlp drliftmsid lyqanpamin |
| 24l | etlkeyspdf vilsnadsqt mkqlvfpssv kkltiksnil drfdfslyns eiqelelytp |
| 30l | niteynplal npkthlisdt dystrflsin lygaqltnqq alvaledvfv rryyeralqg |
| 36l | sfvggyissl vlsdtgitsl nnlvikninp nydsytmsvk yhsndsgqie llkttawetp |
| 42l | tptptptptp dqaaapgdgr vnvedkdlrl lvssevpvna evlinvvsky lynntrvnil |
| 48l | disksllksg slvdvanslk akipylnvvi |
| SEQ ID NO: 28 | |
| 4l | lyqdklisgq nqplapvnrl |
| 6l | igfqtlakfr iedldfelqk kiysstvesa elvnrsavlv ddsvlenhdg eltsvqsdpq |
| l2l | vpapvkilak eqtghtsdfv sgysddnkyy qspyyyndry ympildspti ylknertssd |
| l8l | iglnnyqgwi avgharvnsr vsvfnyratd ellakfnnlp drliftmsid lyqanpamin |
| 24l | etlkeyspdf vilsnadsqt mkqlvfpssv kkltiksnil drfdfslvns eiqelelytp |
| 30l | niteynplal npkthlisdt dystrflsin lygaqltnqq alvaledvfv rryyeralqg |
| 36l | sfvggyissl vlsdtgitsl nnlvikninp nydsytmsvk yhsndsgqie llkttawetp |
| 42l | tptptptptp dqaaapgdgr vnvedkdlrl lvssevpvna evlinvvsky lynntrvnil |
| 48l | disksllksg slvdvanslk akipylnvvi |
| SEQ ID NO: 29 | |
| 4l | lyqdklisgq nqplapvnrl |
| 6l | igfqtlakfr iedldfelqk kiysstvesa elvnrsavlv ddsvlenhdg eltsvqsdpq |
| l2l | vpapvkilak eqtghtsdfv sgysddnkyy qspyyyndry ympildspti ylknertssd |
| l8l | iglnnyqgwi avgharvnsr vsvfnyratd ellakfnnlp drliftmsid lyqanpamin |
| 24l | etlkeyspdf vilsnadsqt mkqlvfpssv kkltiksnil drfdfshins eiqelelytp |
| 30l | niteynplal npkthlisdt dystrflsin lygaqltnqq alvaledvfv rryyeralqg |
| 36l | sfvggyissl vlsdtgitsl nnlvikninp nydsytmsvk yhsndsgqie llkttawetp |
| 42l | tptptptptp tpdqaaapgd grvnvedkdl rllvssevpv naevlinvvs kylynntrvn |
| 48l | ildisksllk sgslvdvans lkakipylnv vi |
| SEQ ID NO: 30 | |
| 4l | lyqdklisgq nqplapvnrl |
| 6l | igfqtlakfr iedldfelqk kiysstvesa elvnrsavlv ddsvlenhdg eltsvqsdpq |
| l2l | vpapvkilak eqtghtsdfv sgysddnkyy qspyyyndry ympildspti ylknertssd |
| l8l | iglnnyqgwi avgharvnsr vsvfnyratd ellakfnnlp drliftmsid lyqanpamin |
| 24l | etlkeyspdf vilsnadsqt mkqlvfpssv kkltiksnil drfdfslvns eiqelelytp |
| 30l | niteynplal npkthlisdt dystrflsin lygaqltnqq alvaledvfv rryyeralqg |
| 36l | sfvggyissl vlsdtgitsl nnlvikninp nydsytmsvk yhsndsgqie llkttawetp |
| 42l | tptpdqaaap gdgrvnvedk dlrllvssev pvnaevlinv vskylynntr vnildisksl |
| 48l | lksgslvdva nslkakipyl nvvi |
| SEQ ID NO: 3l | |
| 4l | lyqdkqisgq nqpldpvnrl |
| 6l | igfqtlakfr iedldfelqk kiysstvesa elvnrsavlv ddsvlenhdg eltsgqsapq |
| l2l | vpapvkilak eqtghtsdfv sgysddnnyc qspyyyndry ympildspti ylknertsrd |
| l8l | igldnyqgwi algharvnsr vsvfnyratd ellakfnnlp drliftmsin lyqanpaiin |
| 24l | etlkeyspdf vilsnadsqt mkqlvfpssv kkltiksnil drfdfslyns eiqelelytp |
| 30l | niteynplal npkthlifda dystrflsin lygaqltnqq alaaledvfv hryyeralqg |
| 36l | sfvggyissl vlsdtgitsl nnlvikninp nydsytmsvk yhsndsgqie llkttawktp |
| 42l | tptptptptp tpdqaaapgd grvsvedkdl rllvssevpv naevlinvvs kylynntrvn |
| 48l | ildisksllk sgslvdvans lkakipylnv vi |
| SEQ ID NO: 32 | |
| 2l | agagigvtlp lvtsnnnhen slnnsssnng snlkvngsvi |
| 6l | stdnlnivat glssnvssqv srqslsssss sestvdskyt akkklttvsg qekeylvstv |
| l2l | yennrkfmpi laydedisyn nyqqsreykd vvygnfpgwd kkvavvhqid nvdlskayas |
| l8l | vaeftpteil knpsapesvk qlyvaldskt mtadvitklv dryqpdylri esvddtsikq |
| 24l | lpdmkyfstv kkvdlggaft tikgvsfptt tqelkissdn iksidplqip esaaiitetv |
| 30l | hdarfteidl sshtdlttdq lqkavnivyk drikerafqg nfaggyiysw nlqntgitsf |
| 36l | ndvsipklnd gtdrfyiayv ayssgnsngt anetitggke psndsqigew wdsssdgwsk |
| 42l | vskvtvtakn gasldynktl teimgflaky pnvktidisl lkfedasktldglkteltnq |
| 48l | ikskygedss yakidfiits qsn |
| SEQ ID NO: 33 | |
| 75 | sklnsi sltdggyrse idlgdgsnfr edfrnfannl seaitdapkd |
| l2l | llrpvpkvev sgliktsstf itpnfkagyy dqvaadgktl kyyqsteyfn nrvvmpilqt |
| l8l | tngtltannr ayddifvdqg vpkfpgwfhd vdkayyagsn gqseylfkew nyyvangspl |
| 24l | ynvypnhhfk qiktiafdap rikqgntdgi nlnlkqrnpd yviingltgd gstlkdlelp |
| 30l | esvkkvsiyg dyhsinvakq ifknvlelef ystnqdnnfg fnplvlgdht niiydlfask |
| 36l | pfnyidltsl elkdnqdnid asklkraysd iyirrrferq mqgywaggyi drylvkntne |
| 42l | knvnkdndtv yaalkdinlh leetythggn tmyrvnenyy pgasayeaer atrdsefqke |
| 48l | ivqr |

Protein M variant polypeptides derived from the various homologs or orthologs include polypeptides that have an amino acid sequence that is substantially identical (e.g., at least 60%, 70%, 75%, 80%, 90%, 95% or 99% identical) to the sequence of any of these MG281 homolog or orthologs (SEQ ID NOs:18-33). Typically, they are capable of generically binding to immunoglobulins. As noted above, some Protein M variants of the invention are soluble polypeptides derived from any of these orthologs or homologs, e.g., polypeptides lacking the N-terminal membrane-spanning regions. Some of these variant polypeptides of the invention can further contain a deletion relative to the wildtype sequence, e.g., deletion of part or all of the C-terminal domain. Some of these variant polypeptides of the invention can further harbor amino acid substitutions at one or more of the conserved residues responsible for hydrogen bond or salt bridge formation. Just like the conserved residues for MG281, such conserved residues in the various homologs or orthologs of MG281 can be readily identified by sequence alignment or other bioinformatics tools. For example, as exemplified herein, the conserved residues for protein Mpn400 from Mycoplasma pneumoniae include, e.g., Tyr149, Ser111, Thr115, Asn456, Tyr459, Ala406, Tyr115, and Ser163. Similarly, the conserved residues for protein MYPE1380 from Mycoplasma penetrans include, e.g., Ala343, Tyr115, and Ser118. In some of these embodiments, the conserved residues in the Protein M homologs or orthologs are replaced via conservative substitutions or with non-polar residues such as phenylalanine.

In some embodiments, the Protein M variant polypeptides have an amino acid sequence that is substantially identical to a Protein M homolog or ortholog sequence selected from SEQ ID NOs:18-33 or fragment thereof. The Protein M variant polypeptides can also contain substitutions at one or more conserved residues for forming hydrogen bonds or salt bridge with antibodies. In some of these embodiments, the Protein M homolog or ortholog sequence lacks part or all of the N-terminal membrane-spanning region. In some embodiments, the Protein M homolog or ortholog sequence can also have a deletion of part or all of the C-terminal domain. Specific examples of polypeptides derived from the Protein M homologs or orthologs include proteins consisting of a sequence shown in SEQ ID NO:22 or SEQ ID NO:33 (optionally with one or more substitutions at residues Tyr149, Ser111, Thr115, Asn456, Tyr459, Ala406, Tyr115, and Ser163) or proteins consisting of a sequence shown in SEQ ID NO:32 (optionally with one or more substitutions at residues Ala343, Tyr115, and Ser118).

Variants of Protein M or its homologs described herein include fragments as described above, and also polypeptides with altered amino acid sequences due to amino acid substitutions, deletions, or insertions. Variant polypeptides may comprise conservative or non-conservative amino acid substitutions, deletions or additions. Derivatives of Protein M or its homologs described herein are polypeptides which have been altered so as to exhibit additional features not found on the native polypeptide. Examples include fusion proteins.

The invention also provides isolated or recombinant polynucleotide or nucleic acid sequences that encode the immunoglobulin-binging polypeptides or fragments thereof described herein, as well as expression vectors harboring such polynucleotides. The term “polynucleotide” is intended to encompass a singular nucleic acid or nucleic acid fragment as well as plural nucleic acids or nucleic acid fragments, and refers to an isolated molecule or construct, e.g., a virus genome (e.g., a non-infectious viral genome), messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide. A nucleic acid may be provided in linear (e.g., mRNA), circular (e.g., plasmid), or branched form as well as double-stranded or single-stranded forms. A polynucleotide may comprise a conventional phosphodiester bond or a non-conventional bond (e.g., an amide bond, such as found in peptide nucleic acids (PNA)). Vectors harboring the polynucleotides encoding Protein M derived Ig-binding polypeptides of the invention are also encompassed by the invention.

In another aspect, the invention provides various industrial applications of the various Protein M variants and orthologs described herein. For example, these variant polypeptides, as well as their derivatives and immunoglobulin-binding fragments, can be useful in many applications in antibody related fields, e.g., as reagents in purification of antibodies and antigen-binding molecules or fragments. As described herein, antigen-binding molecules broadly encompass any antibodies or antibody fragments described herein. Some of these methods include contacting the Protein M derived protein or an Ig-binding fragment thereof, which is attached to a solid support with a biological sample, or other source of immunoglobulins or antigen-binding molecules, for a time sufficient to allow the immunoglobulins or antigen-binding molecules to bind to the protein or fragment attached to the support, and then eluting the immunoglobulin or molecule. Support includes agarose, polyacrylamide, dextran, cellulose, polysaccharide, nitrocellulose, silica, alumina, aluminum oxide, titania, titanium oxide, zirconia, styrene, polyvinyldifluoride nylon, copolymer of styrene and divinylbenzene, magnetic materials, polystyrene, polymethacrylate ester, derivatized azlactone polymer or copolymer, glass, or cellulose; or a derivative or combination thereof. The support is often in the form of beads or particles, with agarose beads preferred, especially those that are cross-linked and range in size from about 1 to about 300 μm, with about 45 to about 165 μm being preferred. The protein may be linked or coupled to the support via coupling chemistry or covalent tethering. In addition, the protein may be immobilized by chemical or physical means. The protein also may be loaded on a biochip or biosensor.

Many well-known techniques or assays for detecting antibodies or antigen-binding molecules in a biological sample may be employed in the practice of the methods of the present invention. These include, e.g., radio-immunoassays (RIA), enzyme-linked immunosorbent assays (ELISA) assays, enzyme immunoassays (EIA), “sandwich” assays, gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin/avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, etc., immunoprecipitation, competitive binding assays and immunohistochemical staining. These and other assays are described, among other places, in Hampton et al. (Serological Methods, a Laboratory Manual, APS Press, St Paul, Minn. (1990)) and Maddox et al. (J. Exp. Med. 158:1211-1216 (1993)).

Due to their ability to engage B cell receptor, some of the Protein M derived polypeptides of the invention can also be used for promoting B cell proliferation and mitosis. For example, it was found that Protein M lacking the membrane domain (e.g., polypeptides consisting of residues 74-468 of SEQ ID NO:1) can induce proliferation of CD19+ human B cells. Also, in the presence of a large excess of free-floating antibodies, the endotoxin-free Protein M variant (Protein M EF) showed preferential binding to B cell receptor in comparison to free-floating antibody. These Protein M variants and derivatives can all be used as mitogenic reagents. For example, they can be immobilized onto a solid support (e.g., magnetic beads) for eliminating or selecting cells with B-cell receptor.

The invention also provides kits for carrying out the methods disclosed herein. For example, the invention provides kits for use in the purification of antibodies from various biological samples. The kits of the invention typically comprise at least one of the Protein M derived immunoglobulin-binding polypeptide or fragment described herein, and a solid support onto which the immunoglobulin-binding polypeptide or fragment has been or can be immobilized. Any of the solid supports described herein or well known in the art (e.g., cellulose or agarose) for immobilizing proteins or peptides can be used in the kits. The kits can optionally contain other reagents for antibody purification (e.g., solutions for eluting bound antibodies from the solid support). The antibody purification kits can further include packaging material for packaging the reagents and a notification in or on the packaging material. The kits can additionally include appropriate instructions for use and labels indicating the intended use of the contents of the kit. The instructions can be present on any written material or recorded material supplied on or with the kit or which otherwise accompanies the kit.

The following examples are intended to illustrate but not limit the invention.

### Examples

### Example 1. Immunoglobulins Selectively Bind to Proteins in Human Mycoplasma

We investigated mycoplasma infection because it has the features of chronicity and, as an obligatory parasite, is largely confined to the surface of cells. As detailed below, we discovered that some human mycoplasmas produce a protein that binds to immunoglobulins (Igs) with high affinity. This protein, which we refer to as Protein M, has a structure that differs from all others in the Protein Data Bank (PDB).

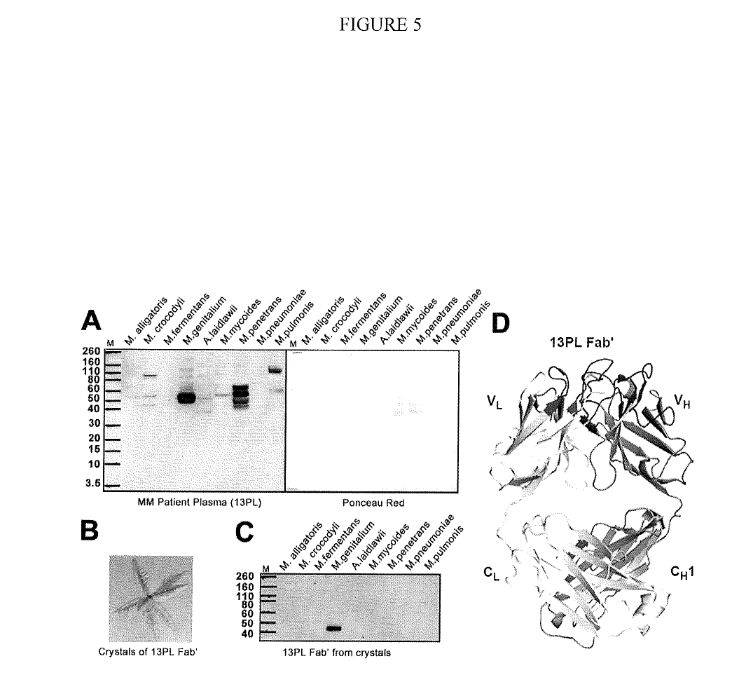

Since we were interested in clonal B-cell proliferation in the context of chronic infections, we investigated whether monoclonal antibodies produced as a result of multiple myeloma react with mycoplasma antigens. We tested the ability of plasma from 20 multiple myeloma patients to bind to total cellular extracts from multiple mycoplasma species, including human pathogens or commensal organisms and others that infect non-human vertebrates. Remarkably, these experiments showed that the antibodies in the plasma all reacted strongly with molecules present in human, but not non-human, pathogenic mycoplasmas (FIG. 5A). The main reactivity was with a protein with an apparent molecular weight of 50 kDa in Mycoplasma genitalium (M. genitalium) and with several proteins with apparent molecular weights of 40-65 kDa in Mycoplasma penetrans (FIG. 5A). We focused our attention on the protein from M. genitalium because it appeared to be more homogeneous as determined by gel electrophoresis (FIG. 5A). The Ig reactivity with the M. genitalium protein was similar for all patients' plasma tested.

To substantiate that the clonal multiple myeloma Ig is the component responsible for binding to the M. genitalium protein, as opposed to a highly reactive protein that co-purifies with it, the Fab′ (fragment antigen-binding) of the primary monoclonal antibody in the plasma of multiple myeloma patient 13PL (13PL Fab′) was highly purified by chromatography followed by crystallization, and its reactivity was studied using dissolved crystals as a source of the antibody (FIG. 5B to 5D). The 13PL Fab′ from the dissolved crystals bound to the same antigen in mycoplasmas as antibodies isolated from whole sera (FIG. 5C). To confirm that the crystals contained an antibody, the x-ray structure was determined at 1.2 Å resolution and only an Fab′ was present (FIG. 5D). To test whether a similar reactivity could be found in blood from non-myeloma normal donors, samples from random donors were studied. The sera from these normal donors also surprisingly reacted with the same mycoplasma proteins as the clonal myeloma Igs, indicating that the ability of human Igs to react with this mycoplasma protein was not confined to those produced in multiple myeloma. We therefore termed the M. genitalium protein that reacts with Igs Protein M.

### Example 2. Protein M Binds Generically to Immunoglobulins

At this point, the possibilities were that Protein M was an antigen to which most people make an antibody, or was a protein that binds to Ig domains or other features that are present in most antibodies. To study these possibilities, we first isolated Protein M using an affinity column constructed from antibody 13PL. The affinity purified Protein M was separated on SDS-PAGE gels followed by Western blot analysis using a different myeloma antibody to confirm the presence of the binding protein. The band on the SDS-PAGE gel corresponding to Protein M was excised and proteomics analysis by mass spectrometry was carried out. These studies showed that Protein M was M. genitalium protein MG281, which is an uncharacterized membrane protein (UniProtKB accession no. P47523) of 556 amino-acids with a predicted trans-membrane domain (residues 16 to 36). Furthermore, homologs of Protein M are present in other mycoplasma strains such as Mycoplasma pneumonia, Mycoplasma iowae and Mycoplasma gallisepticum (UniProtKB data base). Antibodies did not bind to mycoplasma extracts from a Protein M-null M. genitalium mutant, again suggesting that Protein M might be the molecule to which antibodies bind.

To establish that Protein M alone is sufficient for antibody binding, a His-tagged Protein M lacking the membrane-spanning region (recombinant Protein M, residues 37-556) was cloned, expressed in E. coli, and purified by affinity chromatography and size-exclusion chromatography. Western blot analysis of purified Protein M showed that it reacted strongly with the monoclonal Ig from a multiple myeloma patient. In addition, it was found that Protein M also bound to all isotypes of human IgGs as well as mouse, rat, rabbit, goat, and bovine IgGs. To further elucidate the minimum sequence responsible for antibody binding, the Protein M and 13PL IgG complex (mixed in a 1:1.1 molar ratio) was incubated with trypsin for 5 hours. SDS-PAGE gel analysis showed that a truncated protein remained intact after 5 hours as compared to uncomplexed Protein M that was totally digested into smaller fragments. The trypsin-digested Protein M (Protein M TD) was found by mass spectroscopy to contain residues 74 to 482. A His-tagged Protein M TD consisting of residues 74 to 468 (recombinant Protein M TD) was then cloned, expressed in E. coli, and purified by affinity chromatography and size-exclusion chromatography. Protein M and Protein M TD showed similar binding affinities to a panel of Igs or Fabs with K_{d} values in the nM range, as determined using Biolayer Interferometry.

It was also found that Ig binding to Protein M was confined to the Fab domain of the antibody molecule, as shown by Biolayer Interferometry. Since a variety of antibodies with different complementarity determining regions (CDRs) all bind to Protein M, specific interaction with the combining site of the antibody molecule appeared to be excluded. To understand the molecular basis for this interaction, crystal structures of recombinant Protein M TD in complex with two antibody Fabs PGT135 against HIV-1 gp120 with a κ light chain (K_{d}=3.7 nM) and Fab CR9114 against influenza hemagglutinin with a λ light chain (K_{d}=1.9 nM) were determined to 1.65 and 2.50 Å resolution, respectively. Although the 13PL Fab′-Protein M TD complex could not be crystallized, we were able to obtain the structure by electron microscopy, which showed a similar mode of binding. The Protein M structure is very different from any other known Ig binding proteins, such as Protein G, Protein A, and Protein L, or, indeed, any other structures in the Protein Data Bank (www.pdb.org). Protein M TD comprises a large domain (residues 78-440) that includes a leucine-rich repeat (LRR)-like subdomain, and a smaller domain (residues 441-468). Protein M TD binds predominantly to the variable light (V_{L}) domains of both PGT135 Fab and CR9114 Fab, but makes some very limited interactions with the other three Fab domains. The Fab-Protein M TD interactions bury total solvent accessible surface areas of 3590 Å² and 2870 Å² for PGT135 Fab and CR9114 Fab, respectively, mainly from the V_{L} domains of the Fabs. The common interacting positions, which are about two-thirds occupied by hydrophilic residues in both antibodies, are located on one edge of V_{L}. Ten conserved hydrogen bonds and one salt bridge are made from Protein M TD to each Fab V_{L}, including 6 hydrogen bonds to the main chain of V_{L} residues 15, 16, 18, 54 and 77, with the rest to the side chains of V_{L} Arg61, Gln79 and Glu81, almost all of which are highly conserved among human antibodies with both κ and λ light chains. Other residues at the common paratope positions, which make van der Waals contacts and non-conserved H-bonds or salt bridges, are less conserved except for V_{L} Gln37 and Pro59 as well as the completely conserved C_{L} Ser168 and V_{H}1 Ser168. However, some of the non-polar interactions may be conserved even with different amino acids. The N- and C-terminal fragments (residues 37-74 and residues 469-556), which were truncated in Protein M TD as compared to Protein M, are likely disordered as the 3D reconstructions of a Fab in complex with Protein M and Protein M TD using negative-stain electron microscopy are nearly identical.

To determine the scope of Protein M binding to antibodies with different light chain configurations and allotypes, the binding affinities (K_{d}s) of Protein M TD to 24 different light chains in a variety of formats were determined. Because the heavy chains could alter potentially the light-chain conformations, we studied the same germline light chain paired with three different heavy chains. The particular heavy chains made little difference and the K_{d}s varied between 2.1 and 4.8 nM for four V_{H}/V_{L} combinations. Similarly, when the same heavy chain was paired with four different light chains, Protein M binding ranged from 1.8 to 2.3 nM. The effect of allotypic variation was evaluated using five K chain allotypes (κ1, κ2, κ3, κ4 and κ6) and three X chain allotypes (λ2, λ5, and λ11). Allotypic variation had little effect and the K_{d}s for the allotypic variants ranged between 1.0 and 4.8 nM. The preservation of binding affinity in the presence of allotypic variation is to be expected because the critical Protein M contacts are largely conserved among allotypes. To determine whether antigen specificity impacted Protein M, we determined the K_{d}'s for Protein M TD binding to a panel of eight affinity-matured monoclonal antibodies against the same HIV-1 gp120 antigen but with different epitopes and found that the K_{d}'s only varied between 0.7-3.8 nM.

Finally, we assessed the percentage of polyclonal human Igs from the plasma of normal blood donors that was capable of binding to Protein M. After two passages through a column containing Protein M TD immobilized on Ni-NTA matrix at a flow rate of 1 ml/min, greater than 90% of all the Igs were removed, in agreement with our data that showed Protein M binds to all human monoclonal antibodies that we have tested to date.

These structural studies suggested that Protein M should preclude the ability of the antibody to bind to its antigen because it displaces or distorts the CDRs and/or may use its C-terminal domain to sterically block entrance to the antibody combining site. We tested the ability of recombinant Protein M and Protein M TD to block antigen-antibody union for six different antigen-antibody pairs, including two polyclonal auto-antibodies. The monoclonal antibodies used were generated against human influenza virus, HIV-1, human Ebola and mouse Ebola; polyclonal antibodies were purified from Goodpasture's disease patient serum and lupus mouse serum. Blocking of the binding of serum polyclonal antibodies to antigens by Protein M is important because such sera represents a collection of antibodies rather than a single monoclonal species. Prior incubation of the antibodies with Protein M or Protein M TD (in a 1:8 molar ratio) strongly inhibited antibody binding to its cognate antigen, but the order of addition is critical. It was observed that, once antigen-antibody union has occurred for high affinity antigens, Protein M does not disrupt the antibody-antigen complex.

It is important to consider this discovery of a heretofore unknown high affinity Ig binding protein in human M. genitalium in the context of other known Ig binding proteins, such as Protein G, Protein A, and Protein L, which have been invaluable reagents and tools in the antibody field. The Protein M structure is very different from these other Ig binding proteins and is also very different from any other known protein structures. Unlike Protein G, Protein A, and Protein L that all contain multiple, small, Ig binding domains, Protein M has a large domain of 360 residues, which binds principally to antibody V_{L} domains, as well as a leucine-rich repeat (LRR) like motif that faces away from the antibody molecule and may have an as yet uncharacterized function. Importantly, Protein M also contains a 115-residue C-terminal domain that likely protrudes over the antibody combining site. To our knowledge, compared to other known Ig binding proteins, the Protein M TD-antibody Fab buried surface area is the largest.

Protein M binds to antibodies with either κ or λ light chains using conserved hydrogen bonds and salt bridges from backbone atoms and conserved side chains, and some conserved van der Waals interactions, as well as other non-conserved interactions. These conserved interactions provide a structural basis for the broad reactivity with Fvs, Fabs or Igs. In contrast, the primary binding site for Protein G and Protein A is the antibody Fc domain, although secondary lower affinity binding sites include the C_{H}1 domain of IgG for Protein G or V_{H} of the human V_{H}3 gene family for Protein A. Protein L binds only to the V_{L} of most human κ light chains, except for the VκII subgroup. Thus, this new broad-scope, high affinity antibody binding protein, which binds both κ and λ chains, is likely to find a myriad of applications in immunochemistry. In addition to its general use, Protein M may be particularly important for large-scale purification of therapeutic antibodies.

### Example 3. Protein M TD Mutants with Decreased Ig-Binding Activities

Wildtype Protein M binds to immunoglobulins with strong affinity, e.g., dissociation constant in the order of sub-nanomolar range. Due to the strong binding affinity, it is often desirable to obtain variant Protein M molecules with reduced binding affinities. Such molecules are useful when one intends to use Protein M as an affinity purification reagent. This is because the strong binding between Protein M and immunoglobulins as a result of hydrogen bonds can prevent elution of the immunoglobulins, e.g., under standard condition of glycine-HCl buffer (pH 2.7).

Protein M forms nine hydrogen bonds with conserved amino acids residues of the variable light chain region. We decided to mutate six key residues with their non-polar counterparts. We then evaluated the effect of the removal of hydrogen bonds on the binding affinity. These mutants were named PM1 to PM7, which respectively contain mutations at Ser160 with alanine (PM1), Tyr158 with phenylalanine (PM2), Tyr144 with phenylalanine (PM3), Ser 106 with alanine (PM4), Arg 384 with alanine (PM5), Arg384 with Lysine (PM6), and Tyr444 with phenylalanine (PM7).

These mutations were generated using Change-IT multiple mutation Site directed mutagenesis kit. The M. genitalium Protein MG281 (Protein M TD) coding sequence from residues 74 to 482 in a pET-28b (+) vectors that carry a N-terminal His-Tag and a thrombin cleavage site was mutated with mutagenesis primers. The plasmid was amplified in XL1 blue cells. The sequence-confirmed plasmid was then transformed into BL21/DE3 cells, and the overnight starter culture was added to magic media (Life Technology) and cultured for three days at 18° C. in an incubator. N-terminal His-tagged Protein M mutants were purified using Hi-Trap Ni-NTA agarose resin. Protein M mutants were further purified by S-200 size exclusion column after Ni-NTA affinity column.

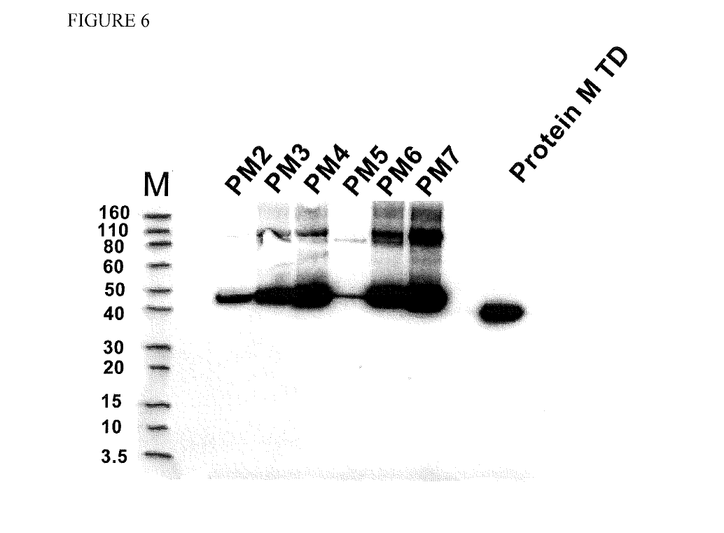

The purified Protein M mutants were then subject to affinity analysis. Western blot analysis of the mutants showed that they reacted with the monoclonal Ig from a multiple myeloma patient. Interestingly, PM2 and PM5 showed decreased binding when compared to the wild type (FIG. 6). PM2 binding constants were then evaluated using Biolayer Interferometry. Human multiple myeloma antibodies, IgG1k, IgG2k (Sigma Aldrich), IgG21 (Sigma Aldrich), IgG3k (Sigma Aldrich), IgG4 (Sigma Aldrich), 4PL (From Mayo Clinic) and 13 PL (From Mayo Clinic), were separately immobilized on a protein G coated biosensors with varying concentrations of the PM2 in solution. The results indicate that dissociation constants (Kd) of PM2 binding to antibodies IgG2κ, IgG2λ, IgG3λ, IgG4λ, 4PL, and 13PL are 9.56 nM, 8.9 nM, 6.6 nM, 1.84 nM, 0.60 nM, and 2.88 nM, respectively. Compared to the sub-nanomolar Kd of unmutated Protein M, the decreased binding constants (ranging from 0.6 nM to 8.9 nM) of PM2 mutant for different IgG subtypes suggest that lack of some hydrogen bonds in Protein M could facilitate elution of IgGs from Protein M under the standard condition of glycine-HCl buffer (pH 2.7).

### Example 4. C-Terminal Domain Truncated Protein M Retains Ig-Binding Activities

We also explored binding activities of Protein M with truncated C-terminal domain. The C-terminal domain of Protein M protrudes over the antibody-combining site and contains 115 amino acid residues. We therefore evaluated the effect of deletion of the C-terminal domain by generating a Protein M TD variant that is truncated at around residue 441.

Specifically, the C-terminal truncated Protein M molecule contains residues 74 to 442. The M. genitalium MG281 (Protein M) coding sequence from resides 74 to 442 was amplified by PCR using primers that annealed to the 5′ and 3′ ends of the gene. The encoded Protein M TD (residues 74 to 442) in a pET-28b (+) vector that carry a N-terminal His-Tag and a thrombin cleavage site protein was expressed and purified as follows. The plasmid was amplified in XL1 blue cells. The sequence confirmed plasmid was transformed into BL21/DE3 cells, and the overnight starter culture was added to magic media (Life Technology) and cultured for three days at 18° C. in an incubator. N-terminal His-tagged Protein M TD truncated version was purified using Hi-Trap Ni-NTA agarose resin. The Protein M mutant was further purified by S-200 size exclusion column after Ni-NTA affinity column.

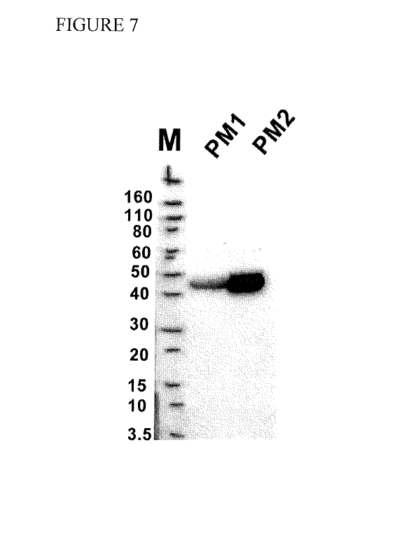

Binding activities of the C-truncated Protein M was then examined. It was found that the C-terminal domain deleted Protein M variant retains the binding characteristics of Protein M TD binding with immunoglobulins. However, it also does not interfere with immunoglobulin binding to its cognate antigen. Specifically, Western blot analysis of purified Protein M TD mutant showed that it reacted strongly with the monoclonal Ig from a multiple myeloma patient (Lane 4, FIG. 7). These results suggesting that this Protein M variant could be used as a chaperone to help crystallize antibody-antigen complex. When labeled with appropriate labeling reagent, this Protein M mutant can also be used for staining bound and unbound antibodies in the tissue, as well as a secondary reagent for immunochemistry.

### Example 5. Immunoglobulin-Binding Protein from Mycoplasma pneumonia

No binding was observed between multiple myeloma antibody to M. pneumoniae cell extract in Western blot analysis (Grover et al., Science 343:656-661, 2014). Nevertheless, bioinformatics analysis revealed the presence of a Protein M ortholog named Mpn400 in M. pneumoniae. This suggests that expression of Mpn400 may be restricted. Mpn400 is the closest ortholog of Protein M (UniProtKB accession no. P75383). Sequences of the two proteins are 56% identical, 70% positive, and have 5 small gaps. This similarity is good, but not exceptional relative to the average similarity of orthologous M. genitalium and M. pneumonia proteins. However, all the contact residues responsible for hydrogen bonds are present in Mpn400 when compared to Protein M, including Protein M (MG281) residues Y144 (˜Mpn400 Y149), 5106 (˜Mpn400 S111), TI 10 (˜Mpn400 T115), N440 (˜Mpn400 N456), Y444 (˜Mpn400 Y459), A391 (˜Mpn400 A406), Y158 (˜Mpn400 Y115), and 5160 (˜Mpn400 S163).

To further examine activities of this Protein M ortholog, we cloned the full length Mpn400 (residues 41 to 582) (SEQ ID NO:22) lacking the membrane domain. Specifically, the M. pneumonia Protein Mpn400 coding sequence from residues 41 to 582 was amplified from M. pneumonia genomic DNA by PCR using primers that annealed to the 5′ and 3′ ends of the gene. The Mpn400 gene was inserted in a pET-28b (+) vectors that carry an N-terminal His-Tag and a thrombin cleavage site. The plasmid was transformed into BL21/DE3 cells. N-terminal His-tagged Protein M was purified using Hi-Trap Ni-NTA agarose resin. The Mpn400 protein was further purified by S-200 size exclusion column after Ni-NTA affinity column. A shorter Mpn400 protein (residues 75 to 484) (SEQ ID NO:33) was similarly generated and purified.

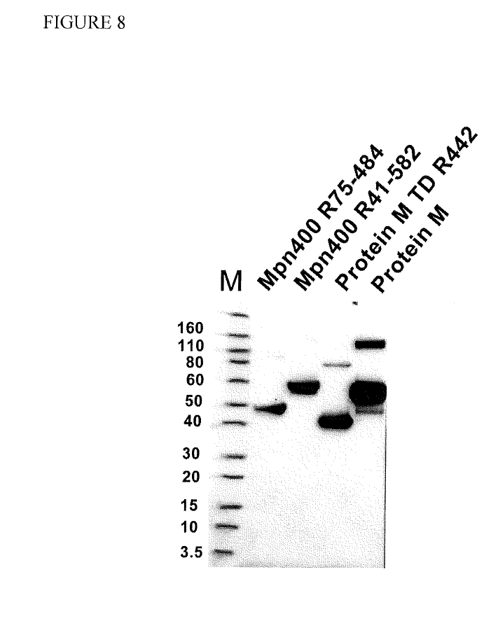

We then tested His-tagged recombinant protein Mpn400 for binding to immunoglobulins. The results indicate that that Mpn400 is sufficient for antibody binding in Western blot analysis (FIG. 8). Also, binding affinities for different antibodies were examined with human multiple myeloma antibodies, IgG1k, IgG2k (Sigma Aldrich), IgG21 (Sigma Aldrich), IgG3k (Sigma Aldrich), IgG4 (Sigma Aldrich), 4PL (From Mayo Clinic) and 13 PL (From Mayo Clinic). The antibodies were separately immobilized on a protein G coated biosensors with varying concentrations of the Mpn400 in solution. The Mpn400 binding dissociation constants (kD) to antibodies IgG1κ, IgG2κ, IgG2λ, IgG3κ, IgG4κ, 4PL IgG, and 13PL IgG were determined to be 0.48 nM, 6.23 nM, 13.0 nM, 0.93 nM, 0.43 nM, 0.60 nM, and 0.50 nM, respectively. With the binding dissociation constant ranging from 0.43 to 13 nM, the results indicate that that Mpn400 has antibody-binding profile that is comparable to that of some Protein M mutants described herein.

### Example 6. Immunoglobulin-Binding Proteins from Mycoplasma penetrans

Using an affinity column constructed from multiple myeloma antibody 13PL, we isolated proteins from the cell extracts of M. penetrans. The affinity-purified M. penetrans protein was separated on SDS-polyacrylamide gel electrophoresis (SDS-PAGE) followed by Western blot analysis with a different myeloma antibody (4PL) to confirm the presence of the binding protein. The corresponding band on the SDS-PAGE was excised, and was identified by mass spectrometry, which revealed it to be MYPE1380, a protein of unknown function. The putative uncharacterized protein MYPE1380 (UniProtKB accession no. Q8EWR5) has 503 amino acids with no predicted transmembrane domain.

We then cloned the full length MYPE1380 (41 to 503) (i.e., SEQ ID NO:32) lacking the membrane domain. Specifically, the M. penetrans Protein MYPE1380 coding sequence from residues 41 to 503 was amplified from M. penetrans genomic DNA by PCR using primers that annealed to the 5′ and 3′ ends of the gene. The MYPE1380 gene was inserted in a pET-28b (+) vectors that carry an N-terminal His-Tag and a thrombin cleavage site. The plasmid was transformed into BL21/DE3 cells. N-terminal His-tagged MYPE1380 was purified using Hi-Trap Ni-NTA agarose resin. Protein MYPE1380 was further purified by S-200 size exclusion column after Ni-NTA affinity column.

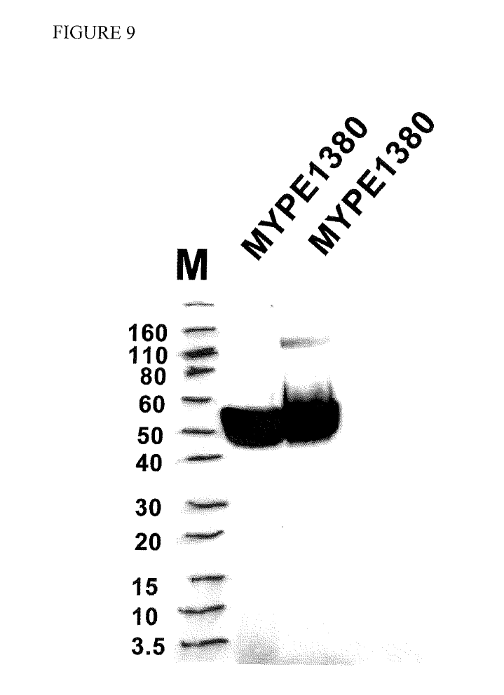

We then examined immunoglobulin-binding activities of this recombinant protein via Western blot analysis. It was found that a His-tagged recombinant MYPE1380 protein (residues 41 to 503) lacking the predicted disordered N-terminal domain is sufficient for antibody binding (FIG. 9). The MYPE1380 binding constant (kD) to human IgG1, IgG2, IgG3 and IgG4 was determined to range from 1.5 to 2.1 nM (data not shown), despite only three hydrogen bonds out of nine involved in Protein M antibody binding being conserved in MYPE1380. The conserved residues are MG281 residue A391 (˜MYPE1380 residue A343), Y158 (˜MYPE1380 residue Y115), and 5160 (˜MYPE1380 residue S118).

### Example 7. Materials and Experimental Protocols

This Example describes some materials and experimental protocols that may be used in the practice of the invention.

IgG purification from multiple myeloma patients or normal blood donor plasma: All myeloma plasma were from The Mayo Clinic collection and normal plasma from The Scripps Research Institute's Normal Blood Donor Service (NBDS). Filtered human plasma samples were loaded onto a HiTrap Protein G HP column with ÄKTAxpress purifier (GE, Pittsburgh, Pa.). The column to which IgG was bound was then washed using phosphate-buffered saline (5 column volumes, PBS). The antibody was eluted with acidic buffer 0.1 M glycine-HCl, pH 2.8 and collected in 1 ml fractions into a tray loaded with 100 μl of 1 M Tris-HCl, pH 8.5 buffer to neutralize the pH.

Preparation and purification of multiple myeloma 13PL Fab′: The Fab′ fragment of human antibody 13PL (IgG1K) was produced by standard protocols. Intact 13PL IgG was digested to (Fab′)₂ with 0.5% (w/w) Immunoglobulin G-degrading enzyme of Streptococcus pyogenes (IdeS) for two hours and followed by reduction to Fab′ by 10 mM L-cysteine for two hours. The protein was purified to homogeneity by a combination of protein A and protein G affinity chromatography.

Crystallization and structural determination of 13PL Fab′: Crystallization experiments were set up using the sitting drop vapor diffusion method. Initial crystallization conditions for the 13PL Fab′ were obtained from robotic crystallization trials using the automated Rigaku Crystalmation system at the Joint Center for Structural Genomics (JCSG, www.jcsg.org). Following the optimization, diffraction quality crystals were obtained by mixing 0.5 μl of the concentrated protein in 7.0 mg/ml in 100 mM sodium acetate, pH 5.5 with 0.5 μl of a reservoir solution containing 0.1 M citric acid, pH 4.0, 1.0 M LiCl₂, 23% (w/v) PEG 6000 at 22° C. The crystals were flashcooled in liquid nitrogen using 25% (v/v) glycerol in mother liquor as cryoprotectant. Diffraction data for the complex crystals were collected at 100K at beamline 11-1, Stanford Synchrotron Radiation Lightsource (SSRL). HKL2000 (HKL Research, Inc.) was used to integrate and scale the data. The P2₁2₁2₁ crystals diffracted to 1.2 Å resolution with Matthews' coefficient (Vₘ) of 2.36 Å₃/Da and 47.4% solvent content. The 13PL Fab′ structure was determined by molecular replacement (MR) using the program Phaser. The initial model for MR was antibody 17/9 Fab (PDB 1HIL). One Fab′ was found in the asymmetric unit. Initial rigid body refinement and restrained refinement were performed using program REFMACS. The structure model was an excellent fit to the electron density maps consistent with the ultra-high resolution of the data except for only two residues (residues 130 and 133) with no density in the heavy chain C_{H}1 130-loop (127 to 133), which usually has poor to no electron density in most Fab crystal structures. Based on the electron density maps (2Fₒ-F_{c} and Fₒ-F_{c}), probable amino acid sequences of V_{L} and V_{H} were incorporated into the model using the graphics program Coot. A rare disulfide linkage within CDR H3 was found between CysH98 and CysH100c.

Mycoplasma cells and cell culture: The following wild-type mycoplasma strains used in this study were obtained from the American Type Culture Collection or from the strain collection of the International Organization for Mycoplasmology (IOM), or Gail Cassell's laboratory at the University of Alabama at Birmingham (UAB): Acholeplasma laidlawii PG8 (ATCC 23206), Mycoplasma alligatoris A21JP2 (ATCC 700619), Mycoplasma crocodyli MP145 (ATCC51981), Mycoplasma fermentans PG18 (ATCC 19989-TTR), Mycoplasma genitalium G37 (ATCC 33530), Mycoplasma mycoides subspecies capri strain GM12 (IOM), Mycoplasma pneumoniae M129 (ATCC 29342), Mycoplasma penetrans (ATCC 55252), Mycoplasma pulmonis CT (UAB). The M. genitalium MG281 protein M-null mutant was made via a process of random transposon bombardment that inserted a ˜6 kbp TN4001 transposon containing a tetracycline resistance marker into the coding sequence 61% of the way between the start and termination codons of the gene. To prevent the loss of the transposon during culture, the mutant was cultured in the presence of 10 mg/L tetracycline.

Mycoplasma protein extract: Cells were grown in SP4 medium at 37° C. plus 5% CO₂. Prior to creation of cell extracts to be loaded onto SDS-PAGE, the cells were washed twice and resuspended in a buffer comprised of 272 mM sucrose, 8 mM HEPES pH 7.4, and either 100 mg/L kanamycin or 200 mg/L puromycin. The cells were lysed according to the manufacturer's protocol using lysis buffer from Sigma Aldrich. Nucleic acids were degraded by treatment with DNase and RNase. A 1× Protease inhibitor cocktail (Roche) was added to prevent proteolytic degradation, centrifuged at 20,800 g for 15 min at 4° C.

Sources of antibodies: Human IgG1κ (catalog no. 15154), IgG2κ (catalog no. 15404), IgG2λ (catalog no. 15279), IgG3κ (catalog no. 15654) and IgG4κ (catalog no. 14639) were purchased from Sigma Aldrich. Human IgA (catalog no. I1010) and IgM (catalog no. 18260) were polyclonal antibodies isolated from colostrum and serum, respectively, and also purchased from Sigma Aldrich. Mouse IgG (catalog no. 010-0102), Rat IgG (catalog no. 012-0102), Rabbit IgG (catalog no. 011-0102), Goat IgG (catalog no. 005-0102) and Bovine IgG (catalog no. 001-0102) were purchased from Rockland Immunochemicals Inc. All the peroxidase-labeled secondary antibodies were purchased from Southern Biotech.

Western blot analysis: The protein extracts were separated on Life Technologies NuPAGE Novex 4-12% Bis-Tris mini SDS gels under reducing conditions and transferred to nitrocellulose membranes using Life Technologies iBlot for Western blot analysis. Immunoblotting was performed in 5% non-fat milk using either multiple myeloma patient's plasma/serum (1 to 5000 dilution), purified 13PL IgG (10 μg/ml) antibody obtained from dissolved crystals of 13PL Fab′ protein (10 μg/ml) as primary antibodies, human antibody subtypes (10 μg/ml), or different animal antibodies (10 μg/ml). The secondary goat anti-human IgG antibody (southern biotech catalog no 2040-05) (1 to 1000 dilution) was conjugated to peroxidase. The bands were detected using SuperSignal West Pico and Dura Chemiluminescent Substrate (Thermo Scientific).

Affinity purification of the antibody binding protein: The 13PL multiple myeloma antibody was conjugated covalently to Protein A/G agarose bead resin using disuccinmidyl suberate (DSS). The antibody resin was then incubated with the mycoplasma protein extract containing the antibody binding protein. After washing to remove non-bound components of the sample, the immunoglobulin binding protein was recovered by dissociation from the antibody with elution buffer (Pierce Crosslink IP Kit, cat. No 26147).

Mass spectrometry and data analysis: The peptides were trapped with a trapping column (Zorbax 300SB-C18, 5 μm×0.3 mm; Agilent) for pre-concentration and desalting using solvent A (99.9% distilled water, 0.1% formic acid). The trapped peptides were then eluted from the trapping column directly onto a reversed phase analytical column (length 14 cm, inner diameter 75 μm, packed with Zorbax SB-C18, 5 μm) using mobile phase solvents (A: 99.9% distilled water, 0.1% formic acid and B: 99.9% acetonitrile, 0.1% formic acid) with the gradient. The eluent was introduced into the linear trap quadrupole mass spectrometer from a nano-ion source with a 2-kV electrospray voltage. The analysis method consisted of a full mass spectrometry (MS) scan with a range of 400-2000 m/z followed by data-dependent MS/MS on the three most intense ions from the full MS scan. The raw data from the linear trap quadrupole were searched using M. genitalium FASTA database with the MASCOT search engine. A peptide mass tolerance of 2.0 Da and MS/MS tolerance of 0.8 Da were allowed for peptides with tryptic specificity.

Expression of recombinant Protein M and Protein M TD in E. coli: The M. genitalium Protein MG281 (Protein M) coding sequence from residues 37 to 556 was amplified from M. genitalium genomic DNA by PCR using primers that annealed to the 5′ and 3′ ends of the gene. The Protein M gene was inserted in a pET-28b (+) vectors that carry a N-terminal His-Tag and a thrombin cleavage site. The plasmid was transformed into BL21/DE3 cells. N-terminal His-tagged Protein M was purified using Hi-Trap Ni-NTA agarose resin. For further purification of Protein M without His-tag, the eluent buffer was exchanged to thrombin protease reaction buffer for cleavage with thrombin. After thrombin cleavage, the buffer was exchanged to HisTrap HP 20 ml Ni-NTA binding buffer (50 mM Tris-HCl, pH 8.0, 150 mM NaCl, 10 mM imidazole). The solution was loaded onto the Ni-NTA column with AKTAxpress purifier (GE, Pittsburgh, Pa.) and the flow-through (unbound proteins) was collected and buffer-exchanged into 50 mM Tris-HCl, pH 8.0, 150 mM NaCl, 2% glycerol buffer. Protein M was further purified by S-200 size exclusion column after Ni-NTA affinity column.

The M. genitalium MG281 (Protein M) coding sequence from resides 74 to 468 was amplified by PCR using primers that annealed to the 5′ and 3′ ends of the gene; the encoded Protein M TD protein was expressed and purified as described above for recombinant Protein M.

K_{d} determination: K_{d}'s were determined by Bio-layer interferometry using an Octet Red instrument (ForteBio, Inc.). For 13PL IgG and 4PL IgG binding with recombinant Protein M, 13PLIgG and 4PL IgG at 50 μg/ml in 1× kinetics buffer (1×PBS, pH 7.4, 0.01% BSA, and 0.002% Tween 20) were loaded onto Protein A coated biosensors and incubated with varying concentrations of Protein M in 1× kinetics buffer. All binding data were collected at 30° C. Six concentrations of Protein M were used, with the highest concentration being 50 nM. The K_{d} reported here was determined from the ratio of k_{off} to kₒₙ.

For recombinant Protein M TD binding with Fab′ and IgG of 13P1 and 4PL, Protein M TD at 50 μg/ml in 1× kinetics buffer were loaded onto Ni-NTA coated biosensors and incubated with varying concentrations of IgGs of 13PL Fab′, 4PL Fab′, 13PL IgG and 4PL IgG in 1× kinetics buffer. All binding data were collected at 30° C. Six concentrations of Fab′ and IgG were used, with the highest concentration being 50 nM. The K_{d} reported here was determined from the ratio of k_{off} to kₒₙ.

For human and mouse anti-Ebola antibody KZ52 IgG and 13F6 IgG binding with recombinant Protein M TD, KZ52 IgG and 13F6 IgG at 50 μg/ml in 1× kinetics buffer were loaded onto Protein A coated biosensors and incubated with varying concentrations of Protein M TD in 1× kinetics buffer. All binding data were collected at 30° C. Six concentrations of Protein M TD were used, with the highest concentration being 100 nM. The K_{d} reported here was determined from the ratio of k_{off} to kₒₙ.

For recombinant Protein M TD binding with the His-tagged Fabs from different germlines (primary antibody), the germline Fabs at 50 μg/ml in 1× kinetics buffer were loaded onto Ni-NTA coated biosensors and incubated with varying concentrations of Protein M TD without His-tag in 1× kinetics buffer. All binding data were collected at 30° C. Six concentrations of Protein M TD were used, with the highest concentration being 100 nM. The K_{d} reported here was determined from the ratio of k_{off} to kₒₙ.

For recombinant Protein M TD binding with anti-HIV neutralizing antibody Fabs, Protein M TD at 50 μg/ml in 1× kinetics buffer were loaded onto Ni-NTA coated biosensors and incubated with varying concentrations of Fabs without His-tag in 1× kinetics buffer. All binding data were collected at 30° C. Six concentrations of Fabs were used, with the highest concentration being 100 nM. The K_{d} reported here was determined from the ratio of k_{off} to kₒₙ.

Crystallization and structural determination of Protein M TD in complex with PGT135 Fab: His-tagged Protein M TD and Fab PGT135, a broad neutralizing antibody against HIV envelope glycoprotein gp120 (IgG1κ) (12), were mixed in a 1:1 molar ratio. The mixture solution was incubated overnight at 4° C. before further purification by gel filtration (Superdex 200 column) to remove unbound Protein M TD and Fab. Crystallization experiments were set up using the sitting drop vapor diffusion method. Initial crystallization conditions for the Protein M TD and PGT135 Fab complex were obtained from robotic crystallization trials using the automated Rigaku Crystalmation system at the Joint Center for Structural Genomics (JCSG). Following the optimization, diffraction quality crystals were obtained by mixing 0.5 μl of the concentrated protein (8.4 mg/ml) in 50 mM Tris, pH 7.6, 150 mM NaCl, 2% glycerol, 1 mM DTT and 0.02% NaN₃ with 0.5 μl of a reservoir solution containing 0.16 M NaF and 19% (w/v) PEG 3350 at 22° C. The 11 crystals were flash-cooled in liquid nitrogen using 25% (v/v) glycerol in mother liquor as cryoprotectant. Diffraction data of the complex crystals were collected at 100K at beamline 12-2, Stanford Synchrotron Radiation Lightsource (SSRL). HKL2000 (HKL Research, Inc.) was used to integrate and scale the diffraction data. The P2₁2₁2₁ crystals diffracted to 1.65 Å resolution with Matthews' coefficient (Vₘ) of 2.23 Å₃/Da and 45.0% solvent content.

The structure of the complex of Protein M TD and PGT135 Fab was determined by molecular replacement (MR) using the program Phaser. The initial model for MR was PGT135 Fab (PDB ID 4JM4). One Fab was found in the asymmetric unit. Initial rigid body refinement was performed using program Phoenix, and interpretable electron density was found for the Protein M TD molecule. Using the AutoBuild function from Phenix, most of the Protein M TD residues (˜80%) could be built automatically except for some loop regions. Further model rebuilding was preformed using the graphics program Coot and refined with Phenix.

Crystallization and structural determination of Protein M TD in complex with CR9114 Fab: His-tagged Protein M TD and the Fab of CR9114, a broad neutralizing antibody against influenza virus hemagglutinin (IgG1λ2) were mixed in a 1:1 molar ratio. The mixture solution was incubated overnight at 4° C. before further purification by gel filtration (Superdex 200 column) to remove uncomplexed Protein M TD and Fab. Crystallization experiments were set up using the sitting drop vapor diffusion method. Initial crystallization conditions for the Protein M TD and CR9114 Fab (IgG1λ2) complex were obtained from robotic crystallization trials using the automated Rigaku Crystalmation system at the Joint Center for Structural Genomics (JCSG). Following optimization, diffraction quality crystals were obtained by mixing 0.5 μl of the concentrated protein (10.5 mg/ml) in 50 mM Tris, pH 7.6, 150 mM NaCl, 2% glycerol, 1 mM DTT and 0.02% NaN₃ with 0.5 μl of a reservoir solution containing 0.1 M MES pH6.29 and 6% (w/v) MPD at 22° C. The crystals were flash-cooled in liquid nitrogen using 20% (v/v) MPD in mother liquor as cryoprotectant. Diffraction data of the complex crystals were collected at 100K at SSRL beamline 11-1. HKL2000 (HKL Research, Inc.) was used to integrate and scale the diffraction data. The P2₁2₁2₁ crystals diffracted to 2.50 Å resolution with Matthews' coefficient (Vₘ) of 2.19 Å₃/Da and 43.9% solvent content.

The structure of the complex between Protein M TD and CR9114 Fab was determined by molecular replacement (MR) using the program Phaser. The initial model for MR was the refined Protein M TD model from the Protein M TD and PGT135Fab complex and CR9114 Fab (PDB ID 4FQH). One Fab and one Protein M TD were found in the asymmetric unit. Initial rigid body refinement was performed using program Phenix. Further model rebuilding was preformed using the graphics program Coot and refined with Phenix.

Protein M blocking of antibody-antigen union: The antigens studied were the H5 influenza hemagglutinin (influenza strainA/Vietnam/1203/2004 (H5N1)), HIV-1 gp120 (JR-FL gp120 core construct), human Ebola virus glycoprotein (GP), Goodpasture's disease autoimmune antigen collagen 4 alpha 3 (COL4A3) and mouse chromatin. Microtiter polyvinyl plates (96-well, Falcon 3911; Becton Dickinson, Heidelberg, Germany) were each coated separately with antigen (100 ng/well in 25 μl PBS (pH 7.2) and were incubated overnight at 4° C. The plates were washed four times with 1×PBS (Invitrogen #21-040-CV) containing 0.05% Tween 20 (Sigma #P9416). The plates were blocked for 1 h with PBST supplemented with blotto (5% non-fat dry milk; 50 μl per well). As primary antibodies, the monoclonal antibodies human CR9114 IgG, human PGT135 IgG, human KZ52 IgG and mouse 13C6IgG were used at 2 μg/ml and the polyclonal antibodies from human Goodpasture's disease and mouse Lupus disease were used as sera. The binding of these antibodies to their antigens was compared with or without Protein M and Protein M TD. Protein M and Protein M TD were used at molar ratio of 8:1 for binding to the monoclonal antibodies and polyclonal antibodies in the sera. The mixture was incubated for 1 h at 25° C. After washing as described above, 25 μl of goat anti-Human IgG Fc HRP (Southern Biotech#2048-05) (1 to 4000 dilution) in blotto was added, with rocking for 45 mins. Plates were washed four times with 100 μl PBST and once with 100 μl dH₂O. A volume of 50 μl of developer solution (mixture of 6 ml of 0.1 M citrate buffer, pH 2.4, 1.8 μl of 30% hydrogen peroxide and 50 μl of 50 mg/ml ABTS) was added to the wasted plate and incubated at 25° C. with rocking for 30 mins and the absorbance was read at 405 nm.

Protein M does not disrupt preformed high affinity antibody-antigen complexes: The antigens studied were HIV-1 gp120 (JR-FL gp120 core construct) and mouse chromatin. Microtiter polyvinyl plates (96-well, Falcon 3911; Becton Dickinson, Heidelberg, Germany) were each coated separately with antigen at 100 ng/well and 35 ng/well in 25 μl PBS (pH 7.2) and were incubated overnight at 4° C. The plates were washed four times with 1×PBS (Invitrogen #21-040-CV) containing 0.05% Tween 20(Sigma #P9416). The plates were blocked for 1 h with PBST supplemented with blotto (5% non-fat dry milk; 50 μl per well). As primary antibodies, the monoclonal antibodies human PGT135 IgG (2 μg/ml) and polyclonal Lupus mouse plasma were used. The primary antibodies were incubated for 1 h at 25° C. The plates were washed as described above. To assess the binding ability of Protein M with the antibody once high affinity antibody is bound to its cognate antigen, Protein M and Protein M TD were used at molar ratio of 8:1 for the antibodies and titrated down the plate. The mixture was incubated for 1 h at 25° C. After washing as described above, 25 μl of goat anti-Human IgG Fc HRP (Southern Biotech #2048-05) (1 to 4000 dilution) in blotto was added, with rocking for 45 mins. Plates were washed four times with 100 μl PBST and once with 100 μl dH₂O. A volume of 50 μl of developer solution (mixture of 6 ml of 0.1 M citrate buffer, pH 2.4, 1.8 μl of 30% hydrogen peroxide and 50 μl of 50 mg/ml ABTS) was added to the washed plate and incubated at 25° C. with rocking for 30 mins and the absorbance was read at 405 nm.

Electron microscopy and sample preparation: IgG or Fab of antibody b12 was incubated with Protein M and 13PL Fab′ was incubated with Protein M TD for one hour at 4° C. and the resulting complexes were purified by size exclusion chromatography and analyzed by electron microscopy. 3 μl of ˜0.01 mg/ml complex was applied for 5 seconds onto a carbon coated 400 Cu mesh grid that had been glow discharged at 20 mA for 30 seconds, then negatively stained with 1% uranyl formate for 30 seconds. Data were collected using a FEI Tecnai F20 electron microscope operating at 120 keV using an electron dose of 30 e⁻/Å₂ and a magnification of 100,000× that resulted in a pixel size of 1.09 Å at the specimen plane. Images were acquired with a Gatan 4k×4k CCD camera using a nominal defocus range of 500 to 900 nm.

Image processing: Particles were picked automatically using DoG Picker and put into a particle stack using the Appion software package. Initial reference-free 2D class averages were calculated using particles binned by two via the Xmipp Clustering 2D Alignment and sorted into classes. Particles representing the Fab-Protein M complex were selected into a substack, binned by two, and then another round of reference-free alignment was carried out using the Xmipp Clustering and 2D alignment and IMAGIC software, resulting in a template stack of 32 images of 2D class averages. The x-ray structure of a Fab filtered to 20 Å resolution was used as an initial model to refine the 32 2D class averages for 20 iterations. Density not corresponding to the Fab was clearly visible after 3 iterations. Using the map from the 32 class average reconstruction, further refinement was carried out against raw particles binned by 2, for 20 cycles. EMAN was used to generate the final 3D reconstruction from 8,797 particles.

Fitting of crystal structure of Protein M TD in complex with PGT135 Fab into the EM density of Fab b12 in complex with either Protein M or Protein M TD: The crystal structure of Protein M TD in complex with PGT135 Fab was manually fitted into the EM reconstructions of Protein M and Protein M TD both in complex with b12Fab as well as Protein M TD in complex with 13PL Fab′ and refined using the Fit in Map function in UCSF Chimera based on correlation optimization. We chose not to replace the PGT135 Fab with the x-ray structure of the b12 Fab because Protein M appears to fix the Fab elbow angle, which differs from that in the crystal structure. The PGT135 Fab and Protein M TD could be positioned within the envelope without significant parts protruding. The boat-shaped density of the Fab, with a dimple between the constant and variable domains, allowed unambiguous docking of the Fab.

Quantitative removal of serum immunoglobulin using a Protein M affinity column: A Ni-NTA His-Trap HP, 1 ml column (Catalog no. 17-5247-01) was loaded with 10 mg of His-tagged Protein M TD in Tris buffer pH 8.0, 150 mM NaCl coupled with AKTAxpress purifier (GE, Pittsburgh, Pa.). Human polyclonal antibodies from normal blood donors were purified as described above. A total of 5 mg of polyclonal antibodies were passed through the affinity column. The unbound polyclonal antibodies were quantified using UV spectrophotometry.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

1. An isolated or recombinant polypeptide, comprising an amino acid sequence that (a) is substantially identical to Protein MG281 having an amino acid sequence shown in SEQ ID NO:1 or to a Protein MG281 fragment, and (b) has deletion of the C-terminal domain or substitutions of one or more conserved residues for forming hydrogen bonds or salt bridge with antibodies.

2. (canceled)

3. The polypeptide of claim 1, wherein the conserved residues are Ser106, Thr110, Tyr144, Tyr158, Ser160, Asn177, Arg384, Ala391, Asn440, and Tyr444.

4. The polypeptide of claim 1, wherein the conserved residues are substituted with non-polar amino acid residues.

5. The polypeptide of claim 1, wherein the Protein MG218 fragment consists essentially of residues 37-556, residues 37-482, residues 74-482, residues 37-468, residues 74-468, residues 37-442, or residues 74-442 of SEQ ID NO:1.

6. The polypeptide of claim 1, comprising an amino acid sequence that (a) is at least 80%, 90%, 95% or 99% identical to Protein MG281 or fragment thereof, and (b) further contains said deletion or substitutions.

7. The polypeptide of claim 1, comprising an amino acid sequence that is identical to the sequence of Protein MG281 or fragment thereof, except for said deletion or substitutions.

8. The polypeptide of claim 1, consisting essentially of an amino acid sequence that (a) comprises an amino acid substitution at residue Y158 or R384, and (b) that is otherwise identical to residues 74-482 of SEQ ID NO:1.

9. The polypeptide of claim 8, wherein the amino acid substitution is Y158F or R384A.

10. An isolated or recombinant soluble polypeptide that is derived from a protein shown in any one of SEQ ID NOs:18-33, wherein the polypeptide lacks the N-terminal membrane-spanning region and is capable of generically binding to immunoglobulins.

11. The polypeptide of claim 10, consisting essentially of an amino acid sequence that is identical or substantially identical to an amino acid sequence shown in any one of SEQ ID NOs:18-33 minus the membrane-spanning region.

12. The polypeptide of claim 10, consisting essentially of SEQ ID NO:22, 32 or 33.

13. The polypeptide of claim 10, further comprising a deletion of the C-terminal domain.

14. The polypeptide of claim 10, further comprising one or more amino acid substitutions at the conserved residues responsible for hydrogen bond or salt bridge formation.

15. An isolated or recombinant soluble polypeptide, comprising a sequence that (a) is substantially identical to a Protein M homolog or ortholog sequence selected from SEQ ID NOs:18-33 or fragment thereof, and (b) has substitutions at one or more conserved residues for forming hydrogen bonds or salt bridge with antibodies.

16. (canceled)

17. The polypeptide of claim 15, wherein the Protein M homolog or ortholog sequence lacks the N-terminal membrane-spanning region.

18. The polypeptide of claim 15, wherein the Protein M homolog or ortholog sequence has a deletion of the C-terminal domain.

19. The polypeptide of claim 15, wherein the Protein M homolog or ortholog sequence is SEQ ID NO:22 or SEQ ID NO:33, and the conserved residues are Tyr149, Ser111, Thr115, Asn456, Tyr459, Ala406, Tyr115, and Ser163.

20. The polypeptide of claim 15, wherein the Protein M homolog or ortholog sequence is SEQ ID NO:32, and the conserved residues are Ala343, Tyr115, and Ser118.

21. A method of purifying or isolating immunoglobulins or antigen-binding molecules from a biological sample, comprising:
contacting a polypeptide of claim 1 attached to a solid support with the biological sample containing immunoglobulins or antigen-binding molecules for a time sufficient to allow the immunoglobulins or antigen-binding molecules to bind the polypeptide attached to the solid support, and
eluting the immunoglobulin or antigen-binding molecules.

22-26. (canceled)

27. A fusion protein comprising the polypeptide of claim 1.

28. An isolated or recombinant polynucleotide encoding the polypeptide of claim 1.

29. A kit comprising a polypeptide of claim 1 and a solid support to which the polypeptide can be attached.

30-31. (canceled)


SEQUENCE LISTING (33 sequences)

SEQ ID NO: 1 - protein, 556 aa, Mycoplasma genitalium
MQFKKHKNSVKFKRKLFWTIGVLGAGALTTFSAVMITNLVNQSGYALVASGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 2 - protein, 537 aa, Mycoplasma genitalium
HHHHHHSSGLVPRGSHMTNLVNQSGYALVASGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 3 - protein, 211 aa, Homo sapiens
DIEMTQSPSSLSASTGDKVTITCQASQDIAQFLDWYQQRPGDTPKLLIYDASNLAIGVPSRFTGSGSGTDFTFTISSLQPEDIAVYYCQHYDDFPISFGPGTKLETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 4 - protein, 11 aa, Homo sapiens
QASQDIAQFLD

SEQ ID NO: 5 - protein, 7 aa, Homo sapiens
DASNLAI

SEQ ID NO: 6 - protein, 9 aa, Homo sapiens
QHYDDFPIS

SEQ ID NO: 7 - protein, 225 aa, Homo sapiens
AVSLVESGGGTVEPGSTLRLSCAASGFTFGSYAFHWVRQAPGDGLEWVAFISYNGSSKYYANFVKGRFTISRDNSSNTLSLQMNSLKASDTAVYYCARAPDCAQADCHKGAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 8 - protein, 5 aa, Homo sapiens
SYAFH

SEQ ID NO: 9 - protein, 17 aa, Homo sapiens
FISYNGSSKYYANFVKG

SEQ ID NO: 10 - protein, 16 aa, Homo sapiens
APDCAQADCHKGAFGY

SEQ ID NO: 11 - protein, 395 aa, Homo sapiens [sequence omitted]

SEQ ID NO: 12 - protein, 136 aa, Homo sapiens
NFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIR

SEQ ID NO: 13 - protein, 174 aa, Homo sapiens
NELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYEN

SEQ ID NO: 14 - protein, 520 aa, Homo sapiens
TNLVNQSGYALVASGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 15 - protein, 412 aa, Homo sapiens
HHHHHHSSGLVPRGSHMSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKR

SEQ ID NO: 16 - protein, 153 aa, Homo sapiens
HHHHHHSSGLVPRGSHMNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIR

SEQ ID NO: 17 - protein, 191 aa, Homo sapiens
HHHHHHSSGLVPRGSHMNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYEN

SEQ ID NO: 18 - protein, 556 aa, Mycoplasma genitalium
MQFKKHKNSVKFKRKLFWTIGVLGAGALTTFSAVMITNLVNQSGYALVASGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTASTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 19 - protein, 556 aa, Mycoplasma genitalium
MQFKKHKNSVKFKRKLFWTIGVLGAGALTTFSAVMITNLVNQSGYALVVPGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 20 - protein, 556 aa, Mycoplasma genitalium
MQFKKHKNSVKFKRKLFWTIGVLGAGALTTFSAVMITNLVNQSGYALVASGRSGNLGFKLFSTQSPSAEAKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQTVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 21 - protein, 556 aa, Mycoplasma genitalium
MQFKKHKNSVKFKRKLFWTIGVLGAGALTTFSAVMITNLVNQSGYALVASGRSGNLGFKLFSTQSSSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 22 - protein, 542 aa, Mycoplasma pneumoniae
NEVLRLQSGETLIASGRSGNLSFQLYSKVNQNAKSKLNSISLTDGGYRSEIDLGDGSNFREDFRNFANNLSEAITDAPKDLLRPVPKVEVSGLIKTSSTFITPNFKAGYYDQVAADGKTLKYYQSTEYFNNRVVMPILQTTNGTLTANNRAYDDIFVDQGVPKFPGWFHDVDKAYYAGSNGQSEYLFKEWNYYVANGSPLYNVYPNHHFKQIKTIAFDAPRIKQGNTDGINLNLKQRNPDYVIINGLTGDGSTLKDLELPESVKKVSIYGDYHSINVAKQIFKNVLELEFYSTNQDNNFGFNPLVLGDHTNIIYDLFASKPFNYIDLTSLELKDNQDNIDASKLKRAVSDIYIRRRFERQMQGYWAGGYIDRYLVKNTNEKNVNKDNDTVYAALKDINLHLEETYTHGGNTMYRVNENYYPGASAYEAERATRDSEFQKEIVQRAELIGVVFEYGVKNLRPGLKYTVKFESPQEQVALKSTDKFQPVIGSVTDMSKSVTDLIGVLRDNAEILNITNVSKDETVVAELKEKLDRENVFQEIRT

SEQ ID NO: 23 - protein, 467 aa, Mycoplasma iowae
QNTSVNVNNNENINYKTNGTVVTGDKLTFSAVVQQNSNISTQAFISDGTKPVGTYNKEINLGKDSITPKYTSGYVETYLESGDTVSRYSSSEYHNNRTLMPILDTKEHYYTSERTYSEIQKGIYRGWEISTPSINYGEKFSYNASAVLKTVFKQLKQETVSAVQFNLGLSDTSIESINSFLKTNSDIQFVTIKGISQDTDLSKLVLPESVQKLTLLGQRNTINDLKLPSELQEIEIYLGSSLKSIDPLIFPKSANIISDVVMNNTSSVFTEIKLSDSTIDNNSPKLQEAIDDVYTYRIKERAFQGLVPGGYIASWDLTGTKVTSFNNVNIPPLNDGTGRFYIAHVEVKTDGNFGNSQNESIGSKPSNDSQINDWFDWGGGWQKVQEVVVSSSENVSLETATQEIMGFIAKYPNVKKINIVNVKLTDGSTHEQLKDNVIKAITAKYGEESQYKDIEFVLPETVPSPVA

SEQ ID NO: 24 - protein, 467 aa, Mycoplasma gallisepticum
SLYQDKQISGQNQPLAPVNRLIDFQTLAKFRIEDLDFELQKKIYSSTVESAELVNRSAVLVDDSVLENHDGELTSGQSDPQVPAPVKILAKEQTGHTSDFVSGYSDDNKYYQSPYYYNDRVYMPILDSPTIYLKNERTSSDIGLNNYQGWIAVGHARVNSRVSVFNYRATDELLAKFNNLPDRLIFTMLIDLYQANPAIINETLKEYSPDFVILSNADSQTMKQLVFPSSVKKLTIKSNILDRFDFSLVNSEIQELELYTPNLTEYNPLALNPKTHLIFDADYSTRFLSINLYGAQLTNQQALAALEDVFVHRYYERALQGSFVGGYISSLVLSDTGITSLNNLVIKNINPNYDSYTMSVKYHSNDSGQIELLKTTAWKTPTPTPTPDQAAAPGDGRVSVEDKDLRLLVSSEVPVNAEVLINVVSKYLYNNTRVNILDISKSLLKSGSLVDVANSLKAKIPYLNVVI

SEQ ID NO: 25 - protein, 476 aa, Mycoplasma gallisepticum
IYTSVKISNSLYQDKQISGQNQPLAPVNRLIGFQTLAKFRIEDLDFELQKKIYSSTVESAELVNRSAVLVDDSVLENHDGELTSGQSDPQVPAPVKILAKEQTGHTSDFVSGYSDDNKYYQSPYYYNDRVYMPILDSPTIYLKNERTSSDIGLNNYQGWIAVGHARVNSRVSVFNYRATDELLAKFNNLPDRLIFTMSIDLYQANPAMINETLKEYSPDFVILSNADSKTMKQLVFPSSVKKLTIKSNILDRFNFSLVNSEIQELELYTPNLTEYNPLALNPKTHLIFDADYSTRFLSINLYGAQLTNQQALAALEDVFVHRYYERALQGSFVGGYISSLVLSDTGITSLNNLVIKNINPNYDSYTMSVKYHSNDSGQIELLKTTAWKTPTPTPTPDQAAAPGDGRVNVEDKDLRLLVSSEVPVNAEVLINVVSKYLYNNTRVNILDISKSLLKSGSLVDVANSLKAKIPYLNVVI

SEQ ID NO: 26 - protein, 472 aa, Mycoplasma gallisepticum
LYQDKQISGQNQPLAPVNRLIGFQTLAKFRIEDLDFELQKKIYSSTVESAELVNRSAVLVDDSVLENHDGELTSGQSDPQVPAPVKILAKEQTGHTSDFVSGYSDDNKYYQSPYYYNDRVYMPILDSPTIYLKNERTSSDIGLNNYQGWIAVGHARVNSRVSVFNYRATDELLAKFNNLPDRLIFTMSIDLYQANPAMINETLKEYSPDFVILSNADSKTMKQLVFPSSVKKLTIKSNILDRFNFSLVNSEIQELELYTPNLTEYNPLALNPKTHLIFDADYSTRFLSINLYGAQLTNQQALAALEDVFVHRYYERALQGSFVGGYISSLVLSDTGITSLNNLVIKNINPNYDSYTMSVKYHSNDSGQIELLKTTAWKTPTPTPTPTPTPTPDQAAAPGDGRVNVEDKDLRLLVSSEVPVNAEVLINVVSKYLYNNTRVNILDISKSLLKSGSLVDVANSLKAKIPYLNVVI

SEQ ID NO: 27 - protein, 472 aa, Mycoplasma gallisepticum; identical to SEQ ID NO: 26

SEQ ID NO: 28 - protein, 470 aa, Mycoplasma gallisepticum
LYQDKLISGQNQPLAPVNRLIGFQTLAKFRIEDLDFELQKKIYSSTVESAELVNRSAVLVDDSVLENHDGELTSVQSDPQVPAPVKILAKEQTGHTSDFVSGYSDDNKYYQSPYYYNDRVYMPILDSPTIYLKNERTSSDIGLNNYQGWIAVGHARVNSRVSVFNYRATDELLAKFNNLPDRLIFTMSIDLYQANPAMINETLKEYSPDFVILSNADSQTMKQLVFPSSVKKLTIKSNILDRFDFSLVNSEIQELELYTPNLTEYNPLALNPKTHLISDTDYSTRFLSINLYGAQLTNQQALVALEDVFVRRYYERALQGSFVGGYISSLVLSDTGITSLNNLVIKNINPNYDSYTMSVKYHSNDSGQIELLKTTAWETPTPTPTPTPTPDQAAAPGDGRVNVEDKDLRLLVSSEVPVNAEVLINVVSKYLYNNTRVNILDISKSLLKSGSLVDVANSLKAKIPYLNVVI

SEQ ID NO: 29 - protein, 472 aa, Mycoplasma gallisepticum
LYQDKLISGQNQPLAPVNRLIGFQTLAKFRIEDLDFELQKKIYSSTVESAELVNRSAVLVDDSVLENHDGELTSVQSDPQVPAPVKILAKEQTGHTSDFVSGYSDDNKYYQSPYYYNDRVYMPILDSPTIYLKNERTSSDIGLNNYQGWIAVGHARVNSRVSVFNYRATDELLAKFNNLPDRLIFTMSIDLYQANPAMINETLKEYSPDFVILSNADSQTMKQLVFPSSVKKLTIKSNILDRFDFSLVNSEIQELELYTPNLTEYNPLALNPKTHLISDTDYSTRFLSINLYGAQLTNQQALVALEDVFVRRYYERALQGSFVGGYISSLVLSDTGITSLNNLVIKNINPNYDSYTMSVKYHSNDSGQIELLKTTAWETPTPTPTPTPTPTPDQAAAPGDGRVNVEDKDLRLLVSSEVPVNAEVLINVVSKYLYNNTRVNILDISKSLLKSGSLVDVANSLKAKIPYLNVVI

SEQ ID NO: 30 - protein, 464 aa, Mycoplasma gallisepticum
LYQDKLISGQNQPLAPVNRLIGFQTLAKFRIEDLDFELQKKIYSSTVESAELVNRSAVLVDDSVLENHDGELTSVQSDPQVPAPVKILAKEQTGHTSDFVSGYSDDNKYYQSPYYYNDRVYMPILDSPTIYLKNERTSSDIGLNNYQGWIAVGHARVNSRVSVFNYRATDELLAKFNNLPDRLIFTMSIDLYQANPAMINETLKEYSPDFVILSNADSQTMKQLVFPSSVKKLTIKSNILDRFDFSLVNSEIQELELYTPNLTEYNPLALNPKTHLISDTDYSTRFLSINLYGAQLTNQQALVALEDVFVRRYYERALQGSFVGGYISSLVLSDTGITSLNNLVIKNINPNYDSYTMSVKYHSNDSGQIELLKTTAWETPTPTPDQAAAPGDGRVNVEDKDLRLLVSSEVPVNAEVLINVVSKYLYNNTRVNILDISKSLLKSGSLVDVANSLKAKIPYLNVVI

SEQ ID NO: 31 - protein, 472 aa, Mycoplasma gallisepticum
LYQDKQISGQNQPLDPVNRLIGFQTLAKFRIEDLDFELQKKIYSSTVESAELVNRSAVLVDDSVLENHDGELTSGQSAPQVPAPVKILAKEQTGHTSDFVSGYSDDNNYCQSPYYYNDRVYMPILDSPTIYLKNERTSRDIGLDNYQGWIALGHARVNSRVSVFNYRATDELLAKFNNLPDRLIFTMSINLYQANPAIINETLKEYSPDFVILSNADSQTMKQLVFPSSVKKLTIKSNILDRFDFSLVNSEIQELELYTPNLTEYNPLALNPKTHLIFDADYSTRFLSINLYGAQLTNQQALAALEDVFVHRYYERALQGSFVGGYISSLVLSDTGITSLNNLVIKNINPNYDSYTMSVKYHSNDSGQIELLKTTAWKTPTPTPTPTPTPTPDQAAAPGDGRVSVEDKDLRLLVSSEVPVNAEVLINVVSKYLYNNTRVNILDISKSLLKSGSLVDVANSLKAKIPYLNVVI

SEQ ID NO: 32 - protein, 483 aa, Mycoplasma penetrans
AGAGIGVTLPLVTSNNNHENSLNNSSSNNGSNLKVNGSVISTDNLNIVATGLSSNVSSQVSRQSLSSSSSSESTVDSKYTAKKKLTTVSGQEKEYLVSTVYENNRKFMPILAYDEDISYNNYQQSREYKDVVYGNFPGWDKKVAVVHQIDNVDLSKAYASVAEFTPTEILKNPSAPESVKQLYVALDSKTMTADVITKLVDRYQPDYLRIESVDDTSIKQLPDMKYFSTVKKVDLGGAFTTIKGVSFPTTTQELKISSDNIKSIDPLQIPESAAIITETVHDARFTEIDLSSHTDLTTDQLQKAVNIVYKDRIKERAFQGNFAGGYIYSWNLQNTGITSFNDVSIPKLNDGTDRFYIAYVAVSSGNSNGTANETITGGKEPSNDSQIGEWWDSSSDGWSKVSKVTVTAKNGASLDYNKTLTEIMGFLAKYPNVKTIDISLLKFEDASKTLDGLKTELTNQIKSKYGEDSSYAKIDFIITSQSN

SEQ ID NO: 33 - protein, 410 aa, Mycoplasma pneumoniae
SKLNSISLTDGGYRSEIDLGDGSNFREDFRNFANNLSEAITDAPKDLLRPVPKVEVSGLIKTSSTFITPNFKAGYYDQVAADGKTLKYYQSTEYFNNRVVMPILQTTNGTLTANNRAYDDIFVDQGVPKFPGWFHDVDKAYYAGSNGQSEYLFKEWNYYVANGSPLYNVYPNHHFKQIKTIAFDAPRIKQGNTDGINLNLKQRNPDYVIINGLTGDGSTLKDLELPESVKKVSIYGDYHSINVAKQIFKNVLELEFYSTNQDNNFGFNPLVLGDHTNIIYDLFASKPFNYIDLTSLELKDNQDNIDASKLKRAVSDIYIRRRFERQMQGYWAGGYIDRYLVKNTNEKNVNKDNDTVYAALKDINLHLEETYTHGGNTMYRVNENYYPGASAYEAERATRDSEFQKEIVQR